(19) United States
(12) Patent Application Publication
(10) Pub. No.: US 2024/0395404 A1
(43) Pub. Date: Nov. 28, 2024

(54) ARTIFICIAL INTELLIGENCE FOR PRECISION MEDICINE

(71) Applicant: C3.ai, Inc., Redwood City, CA (US)

(72) Inventors: Varun Badrinath Krishna, Hayward, CA (US); Natasha Woods, Menlo Park, CA (US); Nicholas Siebenlist, Bethesda, MD (US); Sina Malekian, Palo Alto, CA (US); Matthew Scharf, New York, NY (US); Sharareh Noorbaloochi, New York, NY (US)

(73) Assignee: C3.ai, Inc., Redwood City, CA (US)

(21) Appl. No.: 18/674,781

(22) Filed: May 24, 2024

Related U.S. Application Data
(60) Provisional application No. 63/504,492, filed on May 26, 2023.

(51) Int. Cl. G16H 50/20

(52) U.S. Cl. CPC G16H 50/20

(57) ABSTRACT

Embodiments provide systems and methods for supporting a medical assessment of a target digital entity. To facilitate the medical assessment, a numerical representation of a target digital entity is generated based on at least a portion of source data associated with the target digital entity, and the numerical representation of the target digital entity is compared to numerical representations of a plurality of digital entities to generate similarity values. Each of the similarity values representing a correspondence between the numerical representations of the target digital entity and the plurality of digital entities. Based on the similarity values, one or more candidate digital entities that are similar to the target digital entity are identified. In some aspects, keywords associated with the target digital entity are used to identify an article associated with a diagnosis or treatment of the target digital entity.

### CROSS-REFERENCE TO RELATED APPLICATIONS

The present application claims the benefit of U.S. Provisional Patent Application Ser. No. 63/504,492, filed May 26, 2023, and entitled “Artificial Intelligence for Precision Medicine,” which is hereby incorporated by reference herein.

### TECHNICAL FIELD

The present disclosure generally relates to medical assessment using an artificial intelligence digital twin and more specifically to AI diagnostics or risk-based assessments for diverse clinical insights.

### BACKGROUND

Over the past decades, technological advancements have improved the ability of users to access large amounts of information across a variety of mediums. For example, in several branches of medicine, such as oncology, medical and other information associated with an individual can be generated and gathered over the individual's lifetime. Such information is being increasingly used to provide tailored interventions for patients or individualized health insurance plans for members.

In the medical field, practitioners (e.g., oncologists) may make treatment decisions based on broad disease population recommendation guidelines. When treating a patient with specific symptoms or attributes, a physician may have access to a database of medical information for multiple patients or of research articles. However, the physician does not have unlimited time to make a treatment recommendation and may use a keyword search to try and identify information from the database containing information for treating the patient or a particular disease of the patient. The results of a physician-generated keyword search can be suboptimal, and the physician may be unable to fully leverage relevant available information when making a treatment decision or recommendation, and in a worst-case scenario, may fail to find information that would otherwise have been useful in determining a course of treatment for a patient, which may negatively impact the patient's overall medical condition.

### SUMMARY

Disclosed herein are AI applications and platform using numerical representations of a digital entities for treatment decisions that can be based on a patient's specific medical history and data of similar patients (e.g., other patients with similar medical conditions that have been successfully and/or unsuccessfully treated), rather than merely relying on general guidelines and practices.

To represent the digital entities in the numerical space, the computing system may parse through information, including medical records, of the digital entity to determine one or more of its attributes and generate a numerical representation based on the attributes. The information may include multiomic data associated with the digital entity and the one or more attributes may be based on the multiomic data. For example, multiomic data may include radiomics, genomics, proteomics, and/or the like. Additionally, or alternatively, the numerical representations are generated based on several individual digital entity parameters, which also include embeddings of text-based parameters, such as drugs, treatments, surgery notes, etc. The embeddings may be generated using natural language processing techniques, such as Bidirectional Encoder Representations from Transformers (BERT), or using domain-specific embeddings approaches (e.g., molBert).

Using a domain-specific embedding for certain types of attributes (or categories), such as medication, drugs, treatment, etc., may more accurately capture the similarities or differences between drugs based on their chemical compositions. Further, the numerical representations of digital entities can be based on encodings generated by computer vision models that detect distinctive features from medical images (e.g., tumor size, location). The computing system may use the numeric representations of the digital entities to find and rank other digital entities (e.g., other digital entities representing other patients) that are similar to a specific digital entity (e.g., a target digital entity) representing a patient being treated by a physician or other healthcare professional.

The precision medicine AI application may indicate similar digital entities, one or more attributes of the similar digital entities, or a combination thereof, to a physician to aid the physician in making a treatment recommendation or decision. Accordingly, the physician may recommend a treatment for a patient (e.g., a patient associated with and/or represented by a particular digital entity) based on successful treatments associated with other digital entities who are most similar (e.g., according to one or more factors, such as demographics, medical conditions, disease stages, and the like) to the particular digital entity.

Embodiments provide systems and methods for artificial intelligence (AI) assisted medical assessment of one or more target digital entities. A digital entity can include one or more data records (e.g., health data records, electronic medical records, and/or other digital data records). In some embodiments, a digital entity is associated with a person (e.g., a patient). For example, the digital entity may represent a person and/or otherwise be associated with the person. In some embodiments, a digital entity may comprise an artificial intelligence digital twin of a patient and/or other person. The data records may include non-static data and represent evolving health states, such as diagnostics and genomics (e.g., transcriptome). The data records may also include data from wearable devices (e.g., smartwatches, heart monitors, etc.) which may be transmitted and received in real-time. The systems and methods described herein may be used by health care providers, insurers, and the like, for early detection of disease, recommendation of preventative health measures, categorization of disease odds and disease risk, and other artificial intelligence-based medical assistance.

In addition to the above-described functionality, the precision medicine AI application may be configured to generate numerical representations for one or more research articles. For example, the research articles may include or correspond to studies of medical conditions and associated treatments. The computing system may match one or more articles to particular digital entities based on similarity measures so that physicians can get quick access to relevant prior studies that can help them make treatment decisions. For example, the one or more articles may be associated with multiomic features of a specific digital entity.

The precision medicine AI application generates numerical representations to streamline and aid treatment decisions. In some aspects, the precision medicine AI application aggregates data from diverse sources and provide physicians with information (e.g., to make treatment decisions) that would otherwise be difficult to aggregate. The precision medicine AI application with use of machine automation of data collection and aggregation limits and reduces human errors made by physicians and increase an overall quality of data relied on by medical professionals. Additionally, or alternatively, the precision medicine AI application can identify digital entities (and/or corresponding information) for comparison based on specific criteria (e.g., disease subgroups), leveraging embeddings that capture similarities between treatments such as between medication or drugs. The use of the numeric representations makes it possible for a physician to receive the information in a limited timeframe, which means the information can be used to produce better patient treatment outcomes. It is noted that while the primary examples disclosed herein relate to application of the disclosed techniques for diagnosis and treatment of medical conditions, in some aspects, the techniques disclosed herein may be applied to other use cases, such as enabling an insurance company to use digital entity similarities to create personalized pricing plans for new customers based on their similarity to other digital entities who are already members of a healthcare plan.

The foregoing has outlined rather broadly the features and technical advantages of the present disclosure in order that the detailed description that follows may be better understood. Additional features and advantages will be described hereinafter which form the subject of the claims of the disclosure. It should be appreciated by those skilled in the art that the conception and specific aspects disclosed may be readily utilized as a basis for modifying or designing other structures for carrying out the same purposes of the present disclosure. It should also be realized by those skilled in the art that such equivalent constructions do not depart from the scope of the disclosure as set forth in the appended claims. The novel features which are disclosed herein, both as to organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the disclosed methods and apparatuses, reference should be made to the embodiments illustrated in greater detail in the accompanying drawings, wherein:

FIG. 1 depicts a block diagram of an example of a system for assessment of a target digital entity in accordance with aspects of the present disclosure.

FIG. 2 depicts a block diagram illustrating an example of determining a similarity between two digital entities in accordance with the present disclosure.

FIG. 3 depicts a flow diagram of an example of a method for medical assessment of a target digital entity in accordance with aspects of the present disclosure.

FIG. 4A depicts a flow diagram of an example of a method for determining a treatment recommendation of a target digital entity using machine learning in accordance with aspects of the present disclosure.

FIG. 4B depicts a flow diagram of an example of a method for generating numerical representations in accordance with aspects of the present disclosure.

FIG. 4C depicts a flow diagram of an example of a method for determining literature recommendations associated with determining the treatment recommendation in accordance with aspects of the present disclosure.

FIG. 4D depicts a flow diagram of an example of a method for determining literature recommendations in accordance with aspects of the present disclosure.

FIG. 4E depicts a flow diagram of an example of a method for determining literature recommendations in accordance with aspects of the present disclosure.

FIG. 5 depicts a flow diagram of an example of a method for generating natural language recommendations using machine learning in accordance with aspects of the present disclosure.

FIG. 6 depicts a flow diagram of an example of a method for generating a summary of machine learning insights generated by machine learning models in accordance with aspects of the present disclosure.

FIG. 7 flow depicts a flow diagram of an example of a method for generating a natural language recommendation based on machine learning insights in accordance with aspects of the present disclosure.

FIG. 8 depicts a flow diagram of an example of a method for generating and explaining similarity values associated with a digital entity and candidate digital entities in accordance with aspects of the present disclosure.

FIG. 9 depicts a flow diagram of an example of a method for identifying candidate digital entities that are similar to a target digital entity and determining an output based on the candidate digital entities using machine learning in accordance with aspects of the present disclosure.

It should be understood that the drawings are not necessarily to scale and that the disclosed embodiments are sometimes illustrated diagrammatically and in partial views. In certain instances, details which are not necessary for an understanding of the disclosed methods and apparatuses or which render other details difficult to perceive may have been omitted. It should be understood, of course, that this disclosure is not limited to the particular embodiments illustrated herein.

### DETAILED DESCRIPTION

The precision medicine AI application described herein uses artificial intelligence (AI) and generates numerical representations to improve medical treatment recommendations. In some aspects, the precision medicine AI application aggregates data from diverse sources (e.g., electronic medical records, wearable devices, journals and other literature, artificial intelligence insights, etc.) and rapidly provide physicians with improved information (e.g., to make treatment decisions) that would otherwise be technically difficult or impossible to provide. The use of machine learning and machine automation of data collection and aggregation may limit or reduce human errors made by physicians and increase an overall quality of data relied on by medical professionals. Additionally, or alternatively, the computing system may identify digital entities (and/or corresponding information) for comparison based on specific criteria (e.g., disease subgroups), leveraging embeddings that capture similarities between treatments and between drugs. The use of the numeric representations makes it possible for a physician to receive the information in a limited timeframe, which means the information can be used to produce better patient treatment outcomes. It is noted that while the primary examples disclosed herein relate to application of the disclosed techniques for diagnosis and treatment of medical conditions, in some aspects, the techniques disclosed herein may be applied to other use cases, such as enabling an insurance company to use digital entity similarities to create personalized pricing plans for new customers based on their similarity to other digital entities who are already members of a healthcare plan.

In one example, the precision medicine AI application is used for medical assessment of a target digital entity. A digital entity can include, for example, one or more data records (e.g., health data records, electronic medical records, journals and other literature). The data records may include non-static data and represent evolving health states, such as diagnostics and genomics (e.g., transcriptome). The data records may also include data from wearable devices (e.g., smartwatches, heart monitors, etc.) which may be transmitted and received in real-time.

In some embodiments, a digital entity is associated with a person (e.g., a patient), and the digital representation may comprise data records. For example, the digital entity may represent the person and/or be associated with a person. In one example, a digital entity and/or the data records may comprise an artificial intelligence digital twin of a patient. An example process includes generating a numerical representation of a target digital entity based on at least a portion of source data associated with the target digital entity. The process also includes comparing the numerical representation of the target digital entity to numerical representations of a digital entities. The process further includes generating similarity values based on the comparing, each of the similarity values representing a correspondence between the numerical representations of the target digital entity and the digital entities. The process includes identifying, based on the similarity values, one or more candidate digital entities that are similar to the target digital entity.

In some implementations, for each digital entity of the digital entities, the source data includes one or more data categories, the one or more data categories include age, weight, vitals, lifestyle, prescriptions, disease status, survival status, tumor grade, tumor volume, tumor location, genetic information, single-nucleotide variant (SNV), copy number variation (CNV), treatments, chemotherapy medicine, chemotherapy dose, surgery details, surgery extent of resection, tumor growth rate, grade, diagnosis, radiology dose, concurrent radiology and chemotherapy, histopathological marker, electronic health record (EHR) information, medical record information, physician notes, lab results, test results, immunizations, medical images and reports, and/or a combination thereof. Additionally, or alternatively, the source data is stored at a data source. The data source may include, but is not limited to, electronic health records, proteome profiling, transcriptome profiling, methylome profiling, copy number variations, simple nucleotide variations, clinical notes, histopathology information, radiology information, or a combination thereof. The data sources may include structured and unstructured data, and the systems and methods described herein may operate on both types of data. For example, models can ingest structured and/or unstructured data to generate recommendations, and the like.

In some implementations of the process, the numerical representation for the target digital entity and each digital entity of the digital entities may include a vector having multiple components. Each component of the multiple components may correspond to one or more data categories of data categories of the source data. In some implementations, at least one component of the multiple components includes multiple sub-components. Each sub-component of the multiple sub-components corresponds to a different data category of the data categories or the same data category of the data categories.

Comparing the numerical representation of the target digital entity to the numerical representations of the digital entities may include. For each component of the multiple components, a component-wise comparison is performed between each component of the numerical representation of the target digital entity and a corresponding component of the numerical representation of each of the digital entities. Comparing the numerical representation of the target digital entity to the numerical representations of the multiple digital entities may also include generating a component similarity value based on the comparison of each component of the multiple components of the numerical representation and generating the similarity value as a composite similarity value based on the component similarity values. In some implementations, each component is associated with a weight value of a set of weight values. In some such implementations, for each digital entity of the digital entities other than the target digital entity, generating the composite similarity value may include, for each component, multiplying the component similarity value associated with the component and the weight value associated with the component. Additionally, the composite similarity value may be generated based on the weighted component similarity values.

In some implementations, the source data includes multiple data categories. In some such implementations, the numerical representation of the target digital entity and each digital entity of the digital entities is generated from one or more values, each of which are numerical representations of one type of data. In one example, the numerical representation of the target digital entity and each digital entity of the digital entities is generated by generating a first value associated with a first category, and generating a second value associated with a second category. The numerical representation may be generated based on the first value and the second value. In some other implementations, the numerical representation may further be generated based on one or more additional values. To illustrate, the first value may be generated based on natural language processing (NLP) representations of drugs, the second value may be generated based on genetic information, and a third value may be generated based on vitals and/or medical images. As an illustrative example of generating a value associated with a category, generating the first value associated with the first category may include identifying first data included in the first category, and providing the first data as an input to a first domain specific model to generate the first value. After identifying first data and prior to identifying the first data, the first data may be cleaned to remove personally identifiable information associated with the digital entity. It is noted that one or more values of other categories may be similarly generated, cleaned, or both. In various implementations, values may include vector values. Accordingly, in one example, the first value may comprise a first vector, the second value may comprise a second vector, and so forth.

In some implementations, the first domain-specific model includes a machine-learning (ML) model. Additionally, or alternatively, generating the first value associated with the first category includes normalizing the first value. A sub-component of the numerical representation of the target digital entity or one of the digital entities may be based on the normalized first value.

In some implementations, the first domain specific model includes an ML model that is configured to generate, for each digital entity of multiple digital entities, a numerical representation of data associated with a category. Additionally, metrics may be computed based on the multiple numerical representations to capture a similarity or other relationship between one or more pairs of digital entities of the multiple digital entities.

In some implementations, generating the second value associated with the second category includes identifying second data included in the second category, and providing the second data as an input to a second domain specific model to generate the second value. Additionally, or alternatively, the first domain specific model may be different from the second domain specific model, or the second domain specific model includes a first ML model. The process may also include generating the numerical representations of the digital entities. For example, generating the numerical representations of the digital entities may include identifying third data (corresponding to the patient) included in a third category, providing the third data as an input to a model to generate a model output representation, and generating the numerical representation of the digital entity based on the model output representation. The model may include a second ML model or a natural language model.

In some implementations, generating the numerical representation for the target digital entity, each digital entity of the digital entities, or both includes determining a time, a date, or both, associated with generation of the numerical representation of the digital entity, the portion of the source data, or a combination thereof. Additionally, a timestamp may be generated based on the time, the date, or both. The numerical representation may be generated based on the portion of the source data and the timestamp.

In some implementations, the process includes generating multiple numerical representations for at least one digital entity of the digital entities. The multiple numerical representations of at least one digital entity may include a first numerical representation of the at least one digital entity and a second numerical representation of the at least one digital entity. For example, the first numerical representation of the at least one digital entity may be associated with a first time and the second numerical representation of the at least one digital entity may be associated with a second time that is different from the first time. Additionally, or alternatively, the first time may correspond to a first state of a medical condition of the target digital entity and the second time may correspond to a second state of the medical condition of the target digital entity.

In some implementations, the process includes determining a time period for generating the numerical representations of the digital entities. In some such implementations, different candidate digital entities of the one or more candidate digital entities are identified based on numerical representations corresponding to different time periods. For example, a target patient represented by the target digital entity may be undergoing treatment for stage 2 of a specific cancer at the present time. A candidate patient represented by a candidate digital entity may have recovered from that cancer at that present time. However, that candidate digital entity may have also had the same type of cancer at stage 2 a year previously. In comparing the target digital entity with the candidate digital entity, we generate numerical representations for the target digital entity based on data at the current time and for the candidate digital entity based on data from one year previously, when the corresponding candidate digital entity had stage 2 cancer. In inferring from computations that the target digital entity is similar to how the candidate digital entity had been a year previously, the target patient's physician may choose to prescribe treatments that worked for the candidate digital entity previously.

In some implementations, the process includes determining a first time associated with the first numerical representation of the target digital entity and determining a second time associated with a second numerical representation of the target digital entity. The second time may be subsequent to the first time. For each digital entity of the digital entities other than the target digital entity, a second comparison may be performed between the numerical representation of the digital entity and the second numerical representation of the target digital entity, and a second similarity value may be generated based on the second comparison. Based on one or more similarity values generated based on the second comparison, at least one candidate digital entity that is similar to the target digital entity may be identified. At least one candidate digital entity may be included in or distinct from one or more candidate digital entities.

In some implementations, the process includes, for a candidate digital entity of the one or more candidate digital entities, determining similarity information that indicates how the candidate digital entity is similar to the target digital entity. For the candidate digital entity, the indication of the one or more candidate digital entities further indicates the similarity value between the candidate digital entity and the target digital entity, the similarity information, a recommendation based on the similarity value, or a combination thereof. For example, the recommendation may be a treatment, a pricing or payment option (e.g., a personalized insurance pricing), or another recommendation.

In some implementations, a literature recommendation (e.g., an article recommendation) may be determined based on the numerical representation of the target digital entity, one or more numerical representations of the one or more candidate digital entities, or a combination thereof. Additionally, or alternatively, an indication of the literature recommendation may be output. In some implementations, determining the literature recommendation may include determining a keyword associated with the target digital entity or the one or more candidate digital entities. For each literature item of a literature items, a distance between the literature item and the keyword may be determined and stored. In some implementations, multiple keywords associated with the target digital entity, or the one or more candidate digital entities may be determined. For each literature item of the literature items, a set of distances between the article and the multiple keywords may be determined. Additionally, for each literature item, based on the set of distances, a composite distance may be determined between the literature item and the multiple keywords. A literature item having the smallest composite distance may be selected as the literature recommendation.

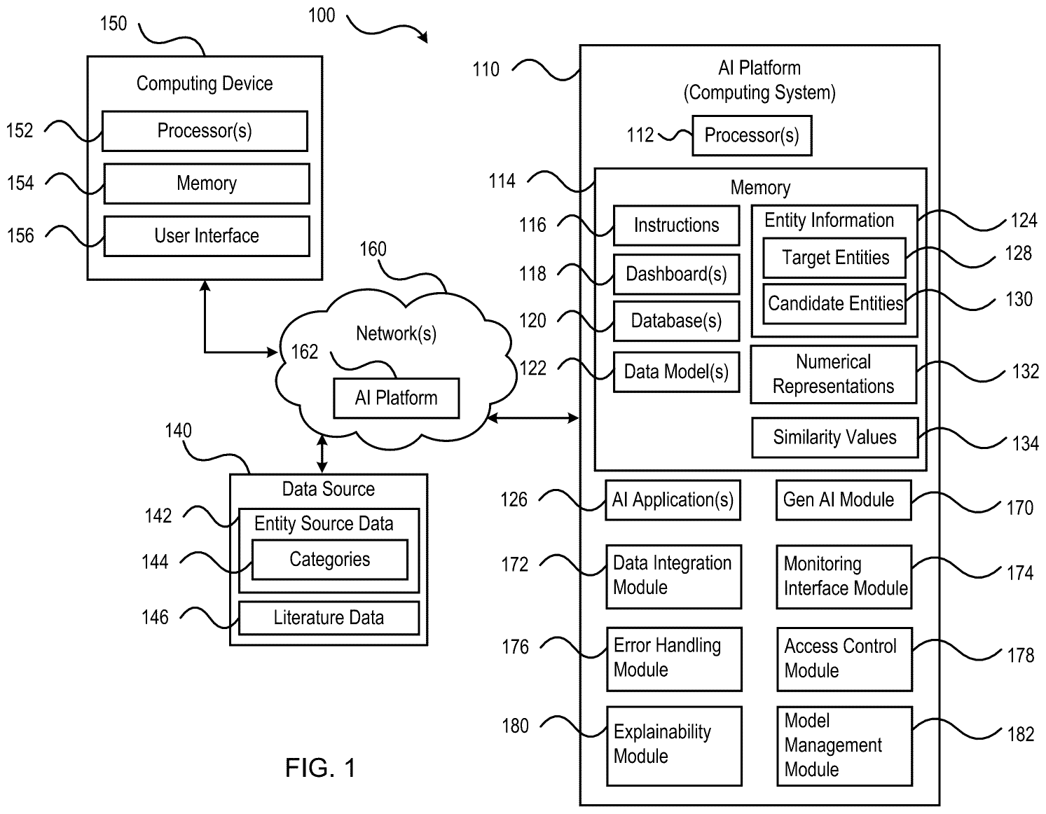

Referring to FIG. 1, a block diagram of an example of a system 100 for medical assessment of a target digital entity in accordance with aspects of the present disclosure is shown. As shown in FIG. 1, the system 100 includes an AI platform 110 (also referred to herein as a computing system and precision medicine AI application), communicatively coupled to one or more computing devices 150 via one or more networks 160. As will be described in more detail below, the AI platform 110 may provide functionality that enables the computing device(s) 150 to help end users of the application with identifying candidate digital entities that are similar to a target digital entity. It is noted that while AI platform 110 is illustrated in FIG. 1 as a standalone device, such as a server, it should be understood that the functionality described herein with respect to the AI platform 110 may be provided by multiple servers (e.g., as a distributed system) or may be provided via a cloud-based implementation, such as cloud-based AI platform 162. In the example of the FIG. 1, the AI platform 110 also includes a generative artificial intelligence sub-system (e.g., generative artificial intelligence module 170), which is described further below.

As shown in FIG. 1, the AI platform 110 includes or operates with one or more processors 112, a memory 114, and one or more AI applications 126. The one or more processors 112 may include one or more microprocessors, microcontrollers, reduced instruction set computers (RISCs), complex instruction set computers (CISCs), graphics processing units (GPUs), data processing units (DPUs), virtual processing units, associative process units (APUs), tensor processing units (TPUs), vision processing units (VPUs), neuromorphic chips, AI chips, quantum processing units (QPUs), cerebras wafer-scale engines (WSEs), digital signal processors (DSPs), application specific integrated circuits (ASICs), field programmable gate arrays (FPGAs), or other discrete circuitry or logic.

The memory 114 may include read only memory (ROM) devices, random access memory (RAM) devices, one or more hard disk drives (HDDs), flash memory devices, solid state drives (SSDs), network attached storage (NAS) devices, cloud-based storage systems, other devices configured to store data in a persistent or non-persistent state, or a combination of different memory devices. The memory 114 may store instructions 116 that, when executed by the one or more processors 112, cause the one or more processors 112 to perform the operations described herein with respect to the AI platform 110. Additionally, the memory 114 may store information in one or more databases 120 and/or as data models 122. The one or more databases 120 may include relational databases, structured databases, unstructured databases, semi-structured databases, or other types of data storage systems and formats. The data models 122 may provide standardized mechanisms for defining the types of data accessible to the AI platform; locations of the data; relationships between different elements or types of data; actions that can initiated from individual elements, batches, or types of data; alternative or secondary storage mechanisms for the data; configurations for backup and normalization schedules; other information that may be used to access and analyze the data stored in the one or more databases 120; or combinations thereof. In some implementations, some or all of the databases 120 may be stored in a digital storage (e.g., a cloud-based digital storage).

In addition to the database(s) 120 and the data models 122, the memory 114 may also store information associated with one or more dashboards 118. The one or more dashboards 118 may correspond to interfaces to access functionality provided by the AI platform 110, such as interfaces that may be presented at or accessible to the computing device 150 for providing information to the AI platform 110 (e.g., a query input) or providing query outputs generated by the AI platform 110 (e.g., a query output) to the computing device 150.

Memory 114 may also be configured to store digital entity information 124, numerical representations 132, and similarity values 134. In some implementations, the digital entity information 124, the numerical representations, the similarity values 134, or a combination thereof may be included in the database 120.

The digital entity information 124 may include information associated with digital entities. For example, the digital entities may include a target digital entity 128 and a candidate digital entity 130. In some implementations, the candidate digital entity 130 is a digital entity that is determined to be similar to the target digital entity 128, as described further herein. The digital entity information 124 may include or indicate, for a digital entity, data, such as age, weight, vitals, lifestyle, prescriptions, disease status, survival status, tumor grade, tumor volume, tumor location, genetic information, single-nucleotide variant, copy number variation, treatments, chemotherapy medicine, chemotherapy dose, surgery details, surgery extent of resection, tumor growth rate, grade, diagnosis, radiology dose, concurrent radiology and chemotherapy, histopathological marker, electronic health record (information, medical record information, or a combination thereof. In some implementations, the data may be arranged or structured based on or according to one or more data categories. Additionally, or alternatively, the digital entity information 124 may include or indicate electronic health records, proteome profiling, transcriptome profiling, methylome profiling, copy number variations, simple nucleotide variations, clinical notes, histopathology information, radiology information, or a combination thereof.

The numerical representations 132 may include numerical representations of one or more digital entities, such as the digital entities. For each digital entity of the digital entities, the numerical representations 132 may be generated based on at least a portion of data associated with the digital entity. In other words, each digital entity may have their own numerical representation.

The similarity values 134 may be generated based on comparisons between different digital entities. For example, the similarity values 134 may be generated based on comparing a numerical representation of the target digital entity to numerical representations of other digital entities of the digital entities. In some implementations, the similarity value between two digital entities may be based on a single numerical representation of each digital entity. It may also be a weighted sum of similarity values across multiple numerical representations of each digital entity, where each representation describes a different data category.

The AI platform 110 may also include one or more AI applications 126. The AI applications 126 may provide functionality for analyzing data hosted by or accessible to the AI platform 110, such as data stored in the one or more database 120 or other types of data (e.g., data stored locally on the computing device 150). For example, the AI applications may be configured to receive, as input, data pertaining to a real-world system, device, scenario, etc. and analyze the input data to derive one or more insights. It is noted that insights obtained via the AI applications 126 may be stored in the one or more databases 120 and information associated with the types of insights that may be derived from each AI application 126 may be stored in the one or more data models 122.

As briefly described above, the AI platform 110 implementing a model driven architecture may be configured to support the functionality disclosed herein. It is noted that a model driven architecture may be used to represent a real-world system (e.g., a computing system, network, the human body, a system of the human body, etc.) using abstraction. For example, using a model driven approach enables a system (e.g., the system 100) or a system component (e.g., the AI platform 110) to be abstracted into different layers, such as applications, analytics, and data structures as non-limiting examples. In such an approach, functionality of the system or system component may be accessed according to the definitions of each layer of abstraction in coordination with behind-the-scenes programming and logic. To illustrate, an application architecture may be abstracted into an interface layer (e.g., a layer associated with the interfaces that interacts with to communicate with the application architecture), an analytics layer (e.g., a layer that defines functionality and analytics that may be accessed via the application architecture), and a type layer. The type layer may be an abstraction layer that defines different inputs that are required to access and/or use the functionality of the analytics layer, permissible data types that may be used with the functionality of the analytics layer, or other parameters and constraints for enabling interaction with the analytics layer via the interface layer. It is noted that the exemplary abstraction layers described above have been provided for purposes of illustration, rather than by way of limitation, and that a model driven architecture in accordance with the present disclosure may include more layers (e.g., an interface layer defining how the interface layer interacts with or communicates with the interface layer, etc.), less layers, or different layers depending on the particular system represented by the model driven architecture. In some aspects, each layer may be represented as a model in which the above-described types of information may be maintained. It is noted that in some aspects, the data models 122 may include models representing all or portions of a model driven architecture supported by the AI platform 110. It is also noted that using a model-driven architecture may enable simplified interaction with the AI platform 110, such as to use AI-based analytics tools provided by the AI applications 126, without requiring extensive programming knowledge and without needing to have a deep understanding of how the AI applications 126 are designed.

The AI platform 110 may be communicatively coupled to the one or more computing devices 150 via the one or more data sources, such as data source 140. In some implementations, data source 140 may include a database, a server, or cloud storage. The data source 140 may include digital entity source data 142, literature data 146, or a combination thereof. The data source 140 may also include environmental data and lifestyle factors.

The digital entity source data 142 may include or correspond to database 120, digital entity information 124, or a combination thereof. In some implementations, the digital entity source data 142 includes one or more data categories 144. The one or more data categories 144 may include age, weight, vitals, lifestyle, prescriptions, disease status, survival status, tumor grade, tumor volume, tumor location, genetic information, single-nucleotide variant, copy number variation, treatments, chemotherapy medicine, chemotherapy dose, surgery details, surgery extent of resection, tumor growth rate, grade, diagnosis, radiology dose, concurrent radiology and chemotherapy, histopathological marker, electronic health record information, medical record information, or a combination thereof. Additionally, or alternatively, the digital entity source data 142 may include or indicate electronic health records, proteome profiling, transcriptome profiling, methylome profiling, copy number variations, simple nucleotide variations, clinical notes, histopathology information, radiology information, or a combination thereof. The literature data 146 may include or indicate one or more articles, reports, documents, the like, or a combination thereof. In some implementations, the literature data 146 may include research articles relating to studies of medical conditions and associated treatments. The digital entity source data 142 may include non-static data and represent evolving health states, such as diagnostics and genomics (e.g., transcriptome). The data records may also include data from wearable devices (e.g., smartwatches, heart monitors, etc.) which may be transmitted and received in real-time, environmental data and lifestyle factors.

As shown in FIG. 1, the AI platform 110 is communicatively coupled to the one or more computing devices 150 via the one or more networks 160. The computing device(s) 150 may correspond to devices (e.g., desktop computing devices, laptop computing devices, tablet computing devices, smartphones, smartwatches, personal digital assistants (PDAs), servers, or other computing devices). The computing device(s) 150 may include one or more processors 152, a memory 154, and one or more interfaces 156. The one or more processors 152 may include one or more types of processors described above with reference to the one or more processors 112 of the AI platform 110 (e.g., CPUs, GPUs, microcontrollers, ASICs, FPGAs, and the like). Similarly, the memory 114 may include one or more types of memory described above with reference to the memory 114 of the AI platform 110 (e.g., RAM, ROM, HDDs, SSDs, and the like). The one or more interfaces 154 may correspond to interfaces that enable interaction with the AI platform 110. For example, the one or more interfaces 154 may include a web browser, a mobile application, or other application executable (e.g., by the one or more processors 152) from the computing device(s) 150 and that provide access to functionality of the AI platform 110. It is noted that although not shown in FIG. 1 for simplicity of the drawing, the computing device 150 may include one or more input/output (I/O) devices, such as a keyboard, a mouse, a touchscreen, a trackpad, a stylus, a microphone, a camera, and the like, to enable inputs to the interfaces 156. Furthermore, the AI platform 110 and the computing device 150 may each include one or more communication interfaces configured to communicatively couple the respective devices to the one or more networks 160 via one or more wired or wireless communication protocols (e.g., an Ethernet protocol, a transmission control protocol/internet protocol (TCP/IP), an institute of electrical and electronics engineers (IEEE) 802.11 protocol, and an IEEE 802.16 protocol, a 3rd Generation (3G) communication standard, a 4th Generation (4G)/long term evolution (LTE) communication standard, a 5th Generation (5G) communication standard, and the like).

To illustrate the functionality provided by the system 100, the AI platform 110 may be configured to generate a numerical representation (e.g., numerical representation 132) of digital entities, such as the target digital entity 128, one or more candidate digital entities 130, or a combination thereof. In some implementations, the numerical representation includes an AI-based digital representation of a patient. The numerical representation of the digital entity may be generated by the AI platform 110 based on multiomic data of or associated with the patient and/or digital representation thereof. For example, the numeric representation of the digital entity can be represented by one or more components representing groups (or categories) of multiomic data. Examples of groups may include “treatments”, “genetics”, “diagnoses” etc. Each group can be separately represented as a vector, called the “component vector”. For example, the “genetics” may be a vector (e.g., list of numbers) containing only quantified genetic values. Additionally, embeddings for drugs or treatments would each contain multiple numbers and would each be “component vectors”. It is noted that a numeric representation may include a vector that is made up of one or more component vectors.

In some implementations, to generate the numeric representation the AI platform 110 may aggregate information from a diverse set of data sources, including but not limited to, electronic health records, proteome profiling, transcriptome profiling, methylome profiling, copy number variations, simple nucleotide variations, clinical notes, histopathology, radiology, or a combination thereof. For example, one or more data sources may be accessible to or integrated in the AI platform 101. The one or more data sources may include one or more digital entity data sources, such as a preprocessed data source. The one or more data sources may include or correspond to the memory 114, the database 120, digital entity information 124, the data source 140, the digital entity source data 142, the categories 144, or a combination thereof. The one or more data sources may include or indicate multiomic data, demographic data (e.g., age), drugs, diagnoses, clinical data (e.g., follow-ups, chemotherapy sessions, radiotherapy sessions), genomic data (e.g., mutations data set, gene copy number data set), other data, or a combination thereof, as illustrative, non-limiting examples.

In some implementations, the one or more data sources include or indicate one or more drugs (e.g., prescriptions) taken by a patient. The AI platform 110 may be configured to generate the numerical representation of the digital entity based on the one or more drugs (e.g., prescriptions) taken by the patient. For example, the AI platform 110 may be configured to perform a natural language processing embedding of the one or more drugs being taken by the patient. To illustrate, the natural language processing may convert drug names to a numerical vector representation using generic embeddings, such as Bidirectional Encoder Representations from Transformers (BERT) or a medical domain-specific embeddings—e.g., molBert, which understand drug compositions.

In some implementations, the one or more data sources include or indicate a diagnosis of a patient. The AI platform 110 may be configured to generate the numerical representation of the digital entity based on the diagnosis of the patient. For example, the AI platform 110 may be configured to perform a natural language processing embedding of the diagnosis of the digital entity using a generic embedding (e.g., BERT) or a medical domain-specific embedding which understand diagnoses.

In some implementations, the one or more data sources include medical images. The images can be encoded by the AI platform 110 into a numerical representation that captures features of the image. For example, the location and size of a tumor as seen in the image can be encoded. Similarity between digital entities can be established by the AI platform 110 based on similarity in those image features, as determined by the distance between the numerical representations. The images can be encoded using approaches that may include machine learning and/or deep learning.

In some implementations, the one or more data sources include or indicate numerical statistics of a digital entity or a condition of the patient. To illustrate, the numerical statistics may include or indicate age, weight, vitals (body temperature, heart rate or pulse rate, respiratory rate, blood pressure), tumor grade, tumor volume, disease status (e.g., “in remission”), survival status, or a combination thereof. The AI platform 110 may be configured to generate the numerical representation of the digital entity based on the numerical statistics. For example, the AI platform 110 may be configured to determine a numerical value of each numerical statistics. It is noted that one or more numerical statistics may be associated with a binary value or a value within a range). To illustrate, disease status may have a value of 1 if the disease is present and a value of 0 if the disease is in remission.

In some implementations, the one or more data sources include or indicate genetic information about the patient. The AI platform 110 may be configured to generate the numerical representation of the digital entity based on the genetic information about the patient. For example, the genetic information may include or indicate a single-nucleotide variant (SNV) or a copy number variation (CNV).

The SNV may include or indicate a number of genes with a single nucleotide mutation that helps characterize the type of disease the digital entity has (e.g., a specific type of cancer). The information can include the type of mutation (missense, stop gain, etc.) and quantified scores of impact on protein structure and function (SIFT score and Polyphen score). To generate the numerical representation of the digital entity based on the genetic information about the digital entity, the numerical representation (or a portion corresponding to the SNV), may include or indicate a gene ID, a digital entity ID, and one or more polyphens—e.g., a vector that includes [Gene ID, Digital entity ID, Polyphen]. In some implementations, the AI platform 110 may identify several genes (e.g., a top 20) responsible for the cancer of a patient. The AI platform 110 may construct a vector V_{P} of polyphons for the genes for the digital entity to be included in the numerical representation. For example, for a digital entity P, the vector V_{P} may be:

V_{P}=(Polyphen_{gene}_{₁},Polyphen_{gene}_{₂}, . . . ,Polyphen_{gene}_{ₙ})

In some implementations, for a first digital entity P₁ and a second digital entity P₂, the AI platform 110 may be configured to compute a distance using, for SNV, V_{P}_{₁} and V_{P}_{₂} (already normalized between 0 and 1). The distance calculated using V_{P}_{₁} and V_{P}_{₂} may be incorporate a patient-patient (or digital entity-digital entity) similarity model, as discussed further herein.

The CNV may include or indicate a number of copies of specific gene segments in the patient's genome and also helps characterize the type of disease the patient has (e.g., specific type of cancer) based on duplication and deletion events. The number of copies could be embedded directly or the difference (or absolute difference) between the number of copies and the expected number of copies could be embedded. To generate the numerical representation of the patient based on the genetic information about the digital entity, the numerical representation (or a portion corresponding to the SNV), may include or indicate a gene ID, a patient ID, and one or more polyphens—e.g., a vector that includes [Gene ID, Patient ID, Copy Number]. In some implementations, the AI platform 110 may identify a number of genes (e.g., a top 20) responsible genes for the cancer. The AI platform 110 may construct a vector V_{P} of copy numbers for the genes for the digital entity. For example, for a patient P, the vector V_{P} may be:

V_{P}=(CopyNumber_{gene}_{₁},CopyNumber_{gene}_{₂}, . . . ,CopyNumber_{gene}_{ₙ})

In some implementations, the AI platform 110 may normalize each of the dimensions of the vector V_{P} over the data points (digital entities): uₚ. Additionally, or alternatively, for the digital entities P₁ and P₂, the AI platform 110 may compute a distance using u_{P}_{₁} and u_{P}_{₂}. The distance calculated using u_{P}_{₁} and u_{P}_{₂} may be incorporate a patient-patient similarity model, as discussed further herein.

In some implementations, the AI platform may identify the copy number that corresponds to a healthy condition (e.g., 2 copies) and numerically represent the digital entities' (e.g., both target and candidate) copy number difference from the healthy copy number. For example, a copy number of 1 could indicate a deletion from the baseline of 2, and the difference is 1. Similarly, greater numbers of copies (above 2) could indicate problematic duplications. Such departures from healthy baselines can be incorporated into the numerical representation of the digital entity.

In some implementations, the one or more data sources include or indicate treatment details (e.g., chemotherapy regimen and radiology dose), surgery details, quantitative measure of the aggressiveness of an individual's cancer, diagnosis, histopathological markers, or a combination thereof. The AI platform 110 may be configured to generate the numerical representation of the patient or corresponding digital entity based on the treatment details, the surgery details, tumor aggression, the diagnosis, the histopathological markers, or a combination thereof.

The AI platform 110 may be configured to use the numerical representation of a digital entity to measure how similar/different one digital entity is from another digital entity. For example, the AI platform 110 may use a numerical representation of a target digital entity (e.g., the target digital entity 128) to determine how similar or different the target digital entity is to one or more other digital entities of the digital entities. In some implementations, the AI platform 110 may receive an indication of the target digital entity and/or target digital entity from the computing device 150. Based on the indication received from the computing device 150, the AI platform 110 may determine how similar the target digital entity is to the one or more other digital entities. The target digital entity and/or target digital entity may be specified by the end user (e.g., physician or insurance agent). Although physicians are described herein, it will be appreciated that the systems and methods described herein can apply to other types of users (e.g., other healthcare providers, insurance agents, etc.).

A similarity (e.g., a similarity value) between two digital entities is conceptually the inverse of the distance between the numerical representations of those digital entities. To determine how similar or different one digital entity (e.g., representing a first patient) is from another digital entity (e.g., representing a second patient), the AI platform 110 may determine a similarity value (e.g., the similarity values 134) based on a first numerical representation of the first digital entity and a second numerical presentation of the second digital entity. It will be appreciated that there could be any number of numerical representations per patient or digital entity (e.g., a numerical representation for each data category) and any number of digital entities and comparisons.

In some implementations, the AI platform 110 is configured to determine an overall digital entity similarity (e.g., the similarity values 134) between two digital entities by computing the distance between two flattened vectors that are a concatenation of multiple component vectors. To illustrate, a first flattened vector may be a concatenation of multiple component vectors of a first digital entity and a second flattened vector may be a concatenation of multiple component vectors of a second digital entity. The distance between the vectors for one digital entity and the corresponding vectors of another digital entity can be computed using a variety of distance measures, such as Manhattan distance, Euclidean distance, cosine distance, etc. If component distances are being individually computed and then later combined, then certain distance measures (like Manhattan distance) may be more convenient to use because the overall patient-patient distance may be a linear function of the component distances. In some implementations, the similarity value may be calculated as a weighted sum of distances between component vectors, where the distance between each component vector is computed separately. For efficiency, those distances can be cached for commonly occurring pairs of vectors. For example, if there are 100 different drug names in a database (e.g., 120), then the pairwise distance measures between their embeddings can be cached. In this manner, the component distance for drugs, between digital entities that use a first drug (e.g., drug 1) and digital entities that use a second drug (e.g., drug 2), would be the same and can be read from the cache. Those distances do not need to be recomputed for every digital entity pair that uses the same drug pair when they have already been computed and cached for the same drug pair.

In some implementations, the component vectors can be normalized/scaled as appropriate so that no individual component distance overshadows other component distances when computing the overall patient-patient distance. For example, a “min-max scaling” can be applied to each component across all digital entities so that all values in the component vectors lie in the [0,1] range.

In some implementations, to calculate a similarity value, the AI platform 110 may compute (and optionally normalize) a component vector of a drug, such as a molBert vector, and calculate an average Manhattan Distance between each unique drug pairing. The similarity value between two vectors (e.g., two different drugs) may be the quantile transformed average Manhattan Distance. Since treatments are a combination of drugs, the average similarity across all the unique pairs is used for the treatment similarity. To further illustrate, chemotherapy treatments often combine multiple drugs and a similarity between treatments may be computed as the average of all the unique pairings between the drugs in each chemotherapy treatment. In some implementations, the AI platform 110 may normalize other types of features (e.g., age, weight, etc.) that are on different scales with each other.

In some implementations, each component or group of components (e.g. vitals, treatments, tumor size, etc.) of a numerical representation of digital entity may have a weight assigned (to the component or the group of components) that contributes to the overall similarity/difference. Those weights may be human-configurable (e.g., a physician can choose which components should play a larger role in computing similarity, such as providing an input via computing device 150), or may be automatically assigned default values that are learned from known physician preferences (which could be determined from past deployments or surveys of physicians). As an illustrative, non-limiting example of weights applied to different components or groups of components, age has a weight of 0.1, disease status has a weight of 0.05, survival status has a weight of 0.03, tumor size has a weight of 0.08, tumor location has a weight of 0.15, chemotherapy medicine (molBERT) has a weight of 0.06, chemotherapy dose has a weight of 0.2, surgery extent of resection has a weight of 0.07, tumor growth rate has a weight of 0.09, grade has a weight of 0.03, diagnosis (BERT) has a weight of 0.04, radiology dose has a weight of 0.06, and concurrent radiology and chemotherapy has a weight of 0.04. As another illustrative, non-limiting example of weights applied to different components or groups of components, age has a weight of 0.5, disease status has a weight of 1, survival status has a weight of 0.8, tumor size has a weight of 0.6, tumor location has a weight of 0.2, chemotherapy medicine (molBERT) has a weight of 0.3, chemotherapy dose has a weight of 0.1, surgery extent of resection has a weight of 1, tumor growth rate has a weight of 0.4, grade has a weight of 0.6, diagnosis (BERT) has a weight of 1, radiology dose has a weight of 0.1, and concurrent radiology and chemotherapy has a weight of 0.1. In this example, the system may divide the sum of the weights to calculate a weighted average.

The AI platform 110 may be configured to associate a numerical representation with a timestamp. It is noted that one or more attributes of a digital entity may change over time. Accordingly, the AI platform 110 may generate numerical representations of the same digital entity at different times and the different numerical representations may be different. At each time stamp (date/time), the numerical representation of the digital entity can be stored at the numerical representations 132 (e.g., in a database) so that comparisons with one digital entity at one point in time can be made with another digital entity at the same or a different point in time. This may be desirable if one digital entity is currently suffering from a disease that another digital entity previously suffered from.

In some implementations, the AI platform 110 may use a K-nearest neighbors (kNN) algorithm to determine a similarity value (e.g., the similarity values 134). For example, the AI platform 110 may normalize input features individually across an entire dataset. For each digital entity, the AI platform 110 may use a value for each of the features, such as the latest value or an average value. For each digital entity, the AI platform 110 may compute the aggregate mean absolute distance from all other digital entities. The AI platform 110 may determine a number, such as 5, of digital entities that are closest to a target digital entity based on the determined distances (e.g., the similarity values 134). The AI platform 110 may output an indication of the number of digital entities that are closest to a target digital entity, provide one or more attributes for each digital entity of the number of digital entities, or a combination thereof.

In some implementations, to determine a similarity value (e.g., the similarity values 134), the AI platform 110 may compute a feature-wise similarity between a first digital entity (Digital entity A) and a second digital entity (digital entity B). For example, the AI platform 110 may compute:

$$Similarity ( A , B ) = \frac{{\sum }_{i} {w}_{i} ( 1 - d ( {f}_{i} ( A ) , {f}_{i} ( B ) ) )}{{w}_{i}} \to Quantile Transform ,$$

where fᵢ is a feature, wᵢ is a weight associated with fᵢ, d(fᵢ(A), fᵢ(B)) is a distance metric used (e.g., Manhattan, Cosine, etc.)—using Manhattan distance as it works for 1-d vectors.

The AI platform 110 may compute a relative feature contribution for similarity as:

$${Δ}_{{f}_{i}} ( A , B ) = \frac{{w}_{{f}_{i}} ( 1 - d ( {f}_{i} ( A ) , {f}_{i} ( B ) ) )}{{\sum }_{i} {w}_{{f}_{i}} ( 1 - d ( {f}_{i} ( A ) , {f}_{i} ( B ) ) )} ,$$

where Δ_{fi}(A, B) is relative contribution for dissimilarity between A and B based on fᵢ.

The AI platform 110 may compute an absolute feature contribution for similarity as:

C_{fi}(A,B)=Similarity(A,B)Δ_{fi}(A,B),

where C_{fi} (A, B) is absolute contribution for similarity between A and B based on fᵢ.

The AI platform 110 may compute group absolute feature contribution for similarity as

G_{I}(A,B)=Σ_{i∈I}C_{fi}(A,B),

where G_{I} (A, B) is a group absolute contribution for all features.

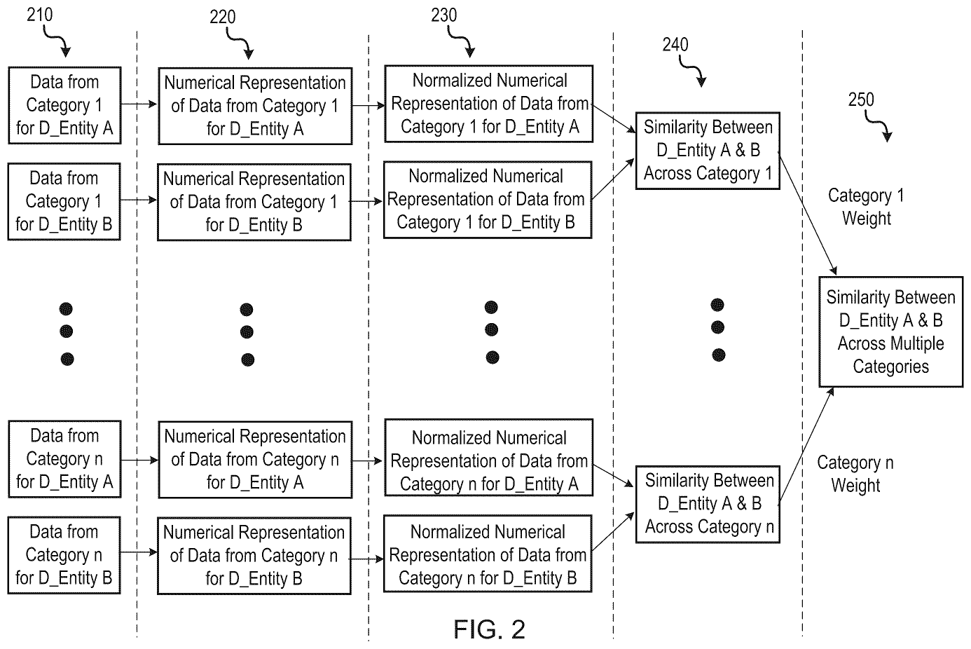

Referring to FIG. 2, FIG. 2 is a block diagram illustrating an example of determining a similarity between two digital entities in accordance with the present disclosure. For example, the similarity (e.g., a similarity value 134) may be determined between digital entities, a first digital entity (Digital entity A) and a second digital entity (Digital entity B) by the AI platform 110 of FIG. 1. The similarity may be determined based on multiple categories, such as categories 1-n, where n is a positive integer greater than 1. Furthermore, the user of the application (e.g., insurance analyst or physician) can choose which data categories are to be used in computing similarity. The data categories can include or exclude categories in a default set of categories proposed by the application. An alternative way to accomplish this would be for the user to provide a non-zero weight for a data category that is to be included and a zero weight for a category that is to be excluded. Data from a category may include or correspond to memory 114, database 120, digital entity information 124, data source 140, digital entity source data 142, or categories 144. The categories may include or correspond to categories 144.

As shown, multiple stages are shown, such as a first stage 210, a second stage 220, a third stage 230, a fourth stage 240, and a fifth stage 250. At the first stage 210, the AI platform 110 may determine, for each of the first digital entity and the second digital entity, data from each category. At the second stage 220, the AI platform 110 generates, for each digital entity, a numerical representation for the data from each category. The numerical representations may include or correspond to numerical representations 132. At the third stage 230, the AI platform 110 normalizes, for each of the digital entities, the numerical presentation for each category. At the fourth stage 240, the AI platform 110 determines, for each category, a similarity (e.g., similarity values 134) between the first digital entity and the second digital entity. At the fifth stage 250, the AI platform 110 uses the similarity values from the fourth stage 240 to determine an overall similarity value (e.g., one or more of the similarity values 134) between the first digital entity and the second digital entity across multiple categories, such as categories 1-n. In some implementations, the AI platform 110 may apply a corresponding weight value to each category similarity value from the fourth stage 240 in order to determine the overall similarity value at the fifth stage 250.

Referring to FIG. 1, the AI platform 110 may determine an explainability component that indicates an extent to which a digital entity is similar to another digital entity. For example, the AI platform 110 may determine the explainability component based on different digital entity attributes of the two digital entities. The AI platform 110 may determine that a first digital entity (digital entity 1) and a second digital entity (digital entity 2) are similar. The AI platform 110 may determine the explainability component based on the overall similarity score (e.g., the similarity value 134) between the first digital entity and the second digital entity. In some implementations, the AI platform 110 may indicate the explainability component as percentage contributions to the overall similarity score from different categories of data. For example, a first percentage (e.g., 50%) for genetic similarity, a second percentage (e.g., 20%) for similarity in diagnosis, a third percentage (e.g., 20%) for similarity in drugs used, and a fourth percentage (e.g., 10%) similarity in treatments received.

In some implementations, the AI platform 110 is configured to preserve privacy of a digital entity such that a numerical representation of the digital entity does not include or indicate identifying information. This may ensure that digital entity data is kept private while attributes used to help identify similar digital entities are revealed in an anonymized fashion to help physicians identify treatments for their digital entities.

In some implementations, the AI platform 110 is configured to generate a numerical representation of literature, such as research articles, relating to studies of medical conditions and associated treatments. For example, the literature may include or correspond to literature data 146. The AI platform 110 may perform a keyword investigation. For example, a keyword investigation may include a keyword search, where an exact match is not necessary, but articles containing words that are synonymous with the keyword (or related to a keyword) would be surfaced. It is noted that some medical keywords map to a similar concept.

The AI platform 110 may use natural language processing to parse through an article, such as the title, abstract, and keywords to create word and sentence embeddings that can be matched to a attributes of a digital entity, such as a diagnosis, treatment, one or more drugs, etc. For example, the AI platform 110 may use a BERT vector embedding of keywords in the literature allows for comparison of terms. The patient-specific words (from diagnoses, treatments, etc.) may be referred to as “keywords” for the article search and a distance is computed between each article (A) and each keyword (K). The AI platform 110 may compute a distance for an (A, K) pair as described herein.

The AI platform 110 may break the title into the words (excluding stopping words): (a₁, a₂, a₃, . . . , a_{N}). The AI platform 110 may compute the vector embedding for each word using a word embedding (like BERT): (Vₐ_{₁}, Vₐ_{₂}, Vₐ_{₃}, . . . , Vₐ_{_{N}}). The AI platform 110 may compute the vector embedding for the keyword: V_{K}; and may calculate the cosine distance between Vₐ_{ᵢ} and V_{K} for all i: (dₐ_{₁}_{,K}, dₐ_{₂}_{,K}, dₐ_{₃}_{,K}, . . . , dₐ_{_{N}}_{,K}). The AI platform may select a word with the least distance: j=argminᵢ dₐ_{ᵢ}_{,K}. The AI platform 110 may generate aⱼ and dₐ_{ⱼ}_{,K} as the closest word, and distance for the (A, K) pair. That distance may be stored (e.g., cached) so that multiple digital entities with the same keyword can leverage precomputed distances from articles.

The AI platform 110 may also compute an overall distance between the digital entity and the article. For example, the AI platform 110 may determine a list (e.g., a vector) f keywords for digital entity P: (K₁, K₂, K₃, . . . , K_{N}). The AI platform 110 may determine (e.g., calculate or read from storage) distances for pairs of (Aᵢ, Kⱼ) for all i, j. Additionally, the AI platform 110 may average the distance over the keywords (Kᵢ), one number for the distance of article-digital entity pair: d_{A}_{ᵢ}_{,P}. The AI platform 110 may identify the relevant articles to show the physicians, one or more articles (e.g., of multiple different articles) having the least average distance d_{A}_{ᵢ}_{,P}. Accordingly, the AI platform 110 may enabling matching articles to digital entities based on similarity measures so that physicians can get quick access to relevant prior studies that can help them make treatment decisions for those digital entities. In some implementations, the AI platform 110 may also conduct exploratory data analysis (EDA) to drive insights from the data to extract features, such as data analysis on the journals, authors, and distribution of literature data over time. In some implementations, generative AI with multimodal models, large language models, and/or the like, may be used to leverage attributes of digital entities to generate one or more queries that are more likely to surface relevant literature results.

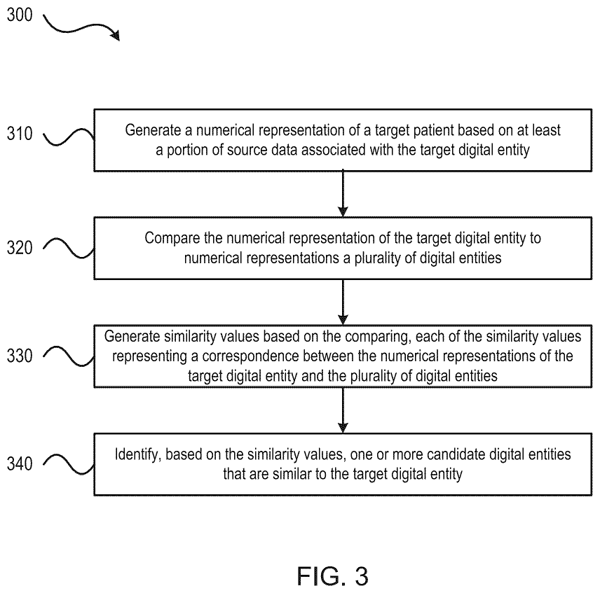

Referring to FIG. 3, a flow diagram of an example of a method 300 for medical assessment of a target digital entity is shown. The method 300 may be performed by one or more computing devices (e.g., the AI platform 110 of FIG. 1), a cloud-based AI platform (e.g., the cloud-based AI platform 162 of FIG. 1), or other implementations. Moreover, steps of the method 300 may be stored as instructions (e.g., the instructions 116 of FIG. 1) that, when executed by one or more processors (e.g., the one or more processors 112 of FIG. 1), cause the one or more processors to perform operations corresponding to the steps of the method 300.

At step 310, the method 300 includes generating a numerical representation of a target digital entity based on at least a portion of source data associated with the target digital entity. The numerical representation of the target digital entity may include or correspond to numerical representations 132. The source data may include or correspond to database 120, digital entity information 124, target digital entity 128, data source 140, digital entity source data 142, categories 144, or a combination thereof.

At step 320, the method 300 includes comparing the numerical representation of the target digital entity to numerical representations of a digital entities. The numerical representation of the digital entities may include or correspond to numerical representations 132.

At step 330, the method 300 includes generating similarity values based on the comparing, each of the similarity values representing a correspondence between the numerical representations of the target digital entity and the digital entities across one or more categories of patient data or digital entity data. The similarity values may include or correspond to similarity values 134.

At step 340, the method 300 includes identifying, based on the similarity values, one or more candidate digital entities that are similar to the target digital entity. The one or more candidate digital entities may include or correspond to candidate digital entity 130.

In some implementations, for each digital entity of the digital entities, the source data includes one or more data categories. For example, the data categories may include or correspond to 144 categories. One or more data categories may include age, weight, vitals, lifestyle, prescriptions, disease status, survival status, tumor grade, tumor volume, tumor location, genetic information, single-nucleotide variant, copy number variation, treatments, chemotherapy medicine, chemotherapy dose, surgery details, surgery extent of resection, tumor growth rate, grade, diagnosis, radiology dose, concurrent radiology and chemotherapy, histopathological marker, electronic health record (information, medical record information, or a combination thereof, as illustrative, non-limiting examples. Additionally, or alternatively, the source data is stored at a data source. For example, the data source may include or correspond to memory 114 or data source 140. The data source may include but is not limited to, electronic health records, proteome profiling, transcriptome profiling, methylome profiling, copy number variations, simple nucleotide variations, clinical notes, histopathology information, radiology information, or a combination thereof.

At step 342 (not shown), the method 300 includes explaining why a target digital entity was deemed to be similar to a candidate digital entity (e.g., through relative contributions of different data categories to the similarity value). In some implementations of the process, the numerical representation for the target digital entity and each digital entity of the digital entities may include a vector having multiple components. Each component of the multiple components may correspond to one or more data categories of a data categories of the source data. In some implementations, at least one component of the multiple components includes multiple sub-components. Each sub-component of the multiple sub-components corresponds to a different data category of the data categories or the same data category of the data categories.

In some implementations, comparing the numerical representation of the target digital entity to the numerical representations of the digital entities may include, for each component of the multiple components, performing a component-wise comparison between each component of the numerical representation of the target digital entity and a corresponding component of the numerical representation of each of the digital entities. Comparing the numerical representation of the target digital entity to the numerical representations of the digital entities may also include generating a component similarity value based on the comparison of each component of the multiple components of the numerical representation, and generating the similarity value as a composite similarity value based on the component similarity values. In some implementations, each component is associated with a weight value of a set of weight values. In some such implementations, for each digital entity of the digital entities other than the target digital entity, generating the composite similarity value may include, for each component, multiplying the component similarity value associated with the component and the weight value associated with the component. Additionally, the composite similarity value may be generated based on the weighted component similarity values.

In some such implementations, the source data includes multiple data categories. Additionally or alternatively, the numerical representation of the target digital entity and each digital entity of the digital entities is generated by generating values associated with each category. In some other implementations, the numerical representation may further be generated based on one or more additional values. To illustrate, the numerical representation may be generated based on the first value may be generated based on NLP representations of drugs, the second value may be generated based on genetic information, and a third value may be generated based on vitals. As an illustrative example of generating a value associated with a category, generating the first value associated with the first category may include identifying first data included in the first category, and providing the first data as an input to a first domain specific model to generate the first value. After identifying first data and prior to identifying the first data, the first data may be cleaned to remove personally identifiable information associated with the digital entity. It is noted that one or more values of other categories may be similarly generated, cleaned, or both.

In some implementations, the first domain specific model includes a machine-learning (ML) model. For example, the ML model may include or correspond to data model 122. Additionally, or alternatively, generating the first value associated with the first category includes normalizing the first value. The numerical representation of the target digital entity or one of the digital entities may be based on the normalized first value.

In some implementations, the first domain specific model includes an ML model that is configured to generate, for each digital entity of multiple digital entities, a numerical representation of data associated with a category. For example, the first domain specific model may include or correspond to data model 122. Additionally, a metric may be computed based on the multiple numerical representations to capture a similarity or other relationship between one or more pairs of digital entities of the multiple digital entities.

In some implementations, generating the second value associated with the second category includes identifying second data included in the second category, and providing the second data as an input to a second domain specific model to generate the second value. For example, the second domain specific model may include or correspond to data model 122. Additionally, or alternatively, the first domain specific model may be different from the second domain specific model, or the second domain specific model includes a first ML model. The process may also include generating the numerical representations of the digital entities. For example, generating the numerical representations of the digital entities may include identifying third data (corresponding to the patient) included in a third category, providing the third data as an input to a model to generate a model output representation, and generating the numerical representation of the digital entity based on the model output representation. The model may include a second ML model or a natural language model.

In some implementations, generating the numerical representation for the target digital entity, each digital entity of the digital entities, or both includes determining a time, a date, or both, associated with generation of the numerical representation of the digital entity, the portion of the source data, or a combination thereof. Additionally, a timestamp may be generated based on the time, the date, or both. The numerical representation may be generated based on the portion of the source data and the timestamp.

In some implementations, the method 300 includes generating multiple numerical representations for the at least one digital entity of the digital entities. The multiple numerical representations of the at least one digital entity may include a first numerical representation of the at least one digital entity and a second numerical representation of the at least one digital entity. For example, the first numerical representation of the at least one digital entity may be associated with a first time and the second numerical representation of the at least one digital entity may be associated with a second time that is different from the first time. Additionally, or alternatively, the first time may correspond to a first state of a medical condition of the target digital entity and the second time may correspond to a second state of the medical condition of the target digital entity.

In some implementations, the method 300 includes determining a time period for generating the numerical representations of the digital entities. In some such implementations, different candidate digital entities of the one or more candidate digital entities are identified based on numerical representations corresponding to different time periods.

In some implementations, the method 300 includes determining a first time associated with the first numerical representation of the target digital entity and determining a second time associated with a second numerical representation of the target digital entity. The second time may be subsequent to the first time. For each digital entity of the digital entities other than the target digital entity, a second comparison may be performed between the numerical representation of the digital entity and the second numerical representation of the target digital entity, and a second similarity value may be generated based on the second comparison. Based on one or more similarity values generated based on the second comparison, at least one candidate digital entity that is similar to the target digital entity may be identified. The at least one candidate digital entity may be included in or distinct from the one or more candidate digital entities.

In some implementations, the method 300 includes, for a first candidate digital entity of the one or more candidate digital entities, determining similarity information that indicates how the first candidate digital entity is similar to the target digital entity. For the first candidate digital entity, the indication of the one or more candidate digital entities further indicates the similarity value between the first candidate digital entity and the target digital entity, the similarity information, a recommendation based on the similarity value, or a combination thereof. For example, the recommendation may be a treatment, a pricing or payment option (e.g., a personalized insurance pricing), or another recommendation.

In some implementations, a literature recommendation (e.g., an article recommendation) may be determined based on the first numerical representation of the target digital entity, one or more numerical representations of the one or more candidate digital entities, or a combination thereof. Additionally, or alternatively, an indication of the literature recommendation may be output. In some implementations, determining the literature recommendation may include determining a keyword associated with the target digital entity or the one or more candidate digital entities. For each literature item of a literature items, a distance between the literature item and the keyword may be determined and stored. For example, the literature items may include or correspond to database 120, data source 140, literature data 146, or a combination thereof. In some implementations, multiple keywords associated with the target digital entity or the one or more candidate digital entities may be determined. For each literature item of the literature items, a set of distances between the article and the multiple keywords may be determined. Additionally, for each literature item, based on the set of distances, a composite distance may be determined between the literature item and the multiple keywords. A literature item having the smallest composite distance may be selected as the literature recommendation.

As can be appreciated from the foregoing, the method 300 provides techniques for medical assessment of a target digital entity. For example, the method 300 provides generation of numeric representations of digital entities which may be used to identify one or more digital entities that are similar to a target digital entity. The use of machine automation of data collection and aggregation may limit or reduce human errors made by physicians and increase an overall quality of data relied on by medical professionals. Additionally, the method 300 may identify digital entities (and corresponding information) for comparison based on specific criteria (like disease subgroups), leveraging embeddings that capture similarities between treatments and between drugs. The use of the numeric representations makes it possible for a physician to receive the information in a limited timeframe, which means the information can be used to produce better digital entity treatment outcomes. In some aspects, an insurance company can use digital entity similarities to create personalized pricing plans for new digital entities based on their similarity to digital entities who are already members of their healthcare plans.

Although the systems and methods illustrated herein are described in some embodiments with reference to physicians and patients, it will be appreciated those are just some embodiments, and other embodiments may be associated with different end users and/or different entities. For example, an end user may be a health insurance agent, and the entity may be an insurance member (e.g., a policyholder or prospective policyholder). Accordingly, a target digital entity would represent an insurance member, and the systems and methods described herein could determine a personalized rate for that insurance member. More specifically, the AI platform 110 could determine, using the numerical representations, other digital entities (e.g., that represent other insurance members) that have similar health as the target digital entity. Rates could be determined by the AI platform 110 for the insurance member based on the rates and/or claims history of the insurance members represented by the similar digital entities.

Referring to FIG. 1, the AI platform 110, in some implementations, may include a generative artificial intelligence module 170. The generative artificial intelligence module 170 may provide features and functionality that are not typically provided by machine learning systems. For example, typical machine learning systems do not handle natural language inputs well and may require expert knowledge by end users in order to be used effectively, which can degrade human computer interactions.

In some implementations, the AI platform 110 and, more specifically, the generative artificial intelligence module 170, may use generative artificial intelligence methodologies and generative artificial intelligence models (e.g., multimodal models, large language models) to provide an intuitive natural language interface between end users and the technologies described herein (e.g., generating numerical representations, determining similar digital entities, and/or other features of the artificial intelligence platform 110). More specifically, the generative artificial intelligence module 170 may allow end users (e.g., healthcare providers, insurance agents) to use natural language inputs (e.g., queries, instruction sets) to obtain natural language results based on determinations of similarities between digital entities.

For example, an input may be “What is the next treatment for my patient?” The generative artificial intelligence module 170 may provide the input to one or more large language models (and/or other machine learning models) which can interpret the query and determine an answer using context provided by the large language models and the underlying features of the AI platform 110. More specifically, the large language models may have been specifically fine-tuned (and/or trained) on some or all of the medical data described herein, which can allow the generative artificial intelligence module 170 to make inferences in response to the natural language input that other machine learning systems would not be able to do For example, the generative artificial intelligence module 170 can leverage the specifically tuned large language models to not only identify similarities, but also generate treatment recommendations. Although in this example the input is a user input, it will be appreciated that inputs can also include system inputs (e.g., machine-readable inputs).

In some implementations, the interface comprises one or more abstraction layers that connects the functionality of the AI platform 110 to various end-user front ends. For example, a front-end may include a graphical user interface that accepts natural language inputs (e.g., search queries input into a search box) which the interface can interpret (e.g., using one or more large language models) to generate a result (e.g., an answer) using the AI platform 110.

The generative artificial intelligence module 170 may also provide interfaces for different types of end users. For example, the generative artificial intelligence module 170 may provide an interface between a patient end-user and features of the artificial intelligence module 170. For example, a patient end user may query the system using “What doctors in my area treat my conditions?” Again, the generative artificial intelligence module 170 may provide the input to the one or more large language models (and/or other machine learning models) which can interpret the query and determine an answer using the context provided by the large language models and the underlying features of the AI platform 110. For example, it may match a numerical representation associated with the end user to similar patients who are being (or have recently been) treated by providers in the geographic location of the end user. Based on the matching, the artificial intelligence module 170 can rank providers based on a combination of their experience with similar patients, their geographical distance to the end user, patient reviews and ratings of their services, etc.

The generative artificial intelligence module 170 may also generate natural language summaries, and other intuitive human-readable summaries, of machine learning insights, recommendations, determinations, and the like, provided by the AI platform 110. For example, a user may provide the query “What guidelines apply to my patient?” The generative artificial intelligence module 170 may not only provide guidelines that apply to the particular patient, but also provide a natural language summary indicating the literature and other documents used to generate the answer. The summary may also include links to those documents. It will be appreciated that the generative artificial intelligence module 170 may interface with some or all of the features of the AI platform 110.

In some implementations, the generative artificial intelligence module 170 may trigger functionality of the AI platform 110. For example, a natural language input processed by one or more trained large language models may trigger a similarity determination between a target digital entity and a other digital entities, generation of recommendations, and so forth.

In some implementations, the large language models may be trained on medical data, as discussed above, but they may also be trained on the numerical representations described herein. This may provide additional context when the generative artificial intelligence module 170 processes the various inputs.

The generative artificial intelligence module 170 may also, in some implementations, generate synthetic numerical representations. For example, a synthetic numerical representation may indicate age, medications, and other features of a numerical representation discussed herein, but not be associated in any way to a real person. This may be useful in clinical trials (e.g., virtual and/or non-virtual clinical trials), privacy enhancing technology, or augmenting existing datasets.

In some implementations, the generative artificial intelligence module 170 can effectively map different conditions and different medications. For example, different geographic locations may have different names for the same medication or the same medical conditions. Since the models have been trained on extensive amounts of medical data, the generative artificial intelligence module 170 can infer relationships between different medications, different medical conditions, and so forth. In one example, a query can include “Where can I find medication X?” The generative artificial intelligence module 170 may infer (e.g., based on the current location of the user) that results relating to medication Y should be presented since they are actually the same medication with different names.

Example inputs that may be processed/answered using generative artificial intelligence may include some or all of the following inputs. For example, an initial input may be “Which patients are most similar to patient ID X?” or “Which patients have had a medical condition similar to condition X?” and the generative artificial intelligence module 170 can return a set of patients similar to the patient having ID X (e.g., based on similarity values). The generative artificial intelligence module 170 may also process follow-up inputs or queries. For example, follow-up inputs to the previous example input can include “Which of those patients responded favorably to drug Y?”, “Which of those patients had an adverse reaction to drug Y?” and/or “Why do you say that patient X is similar to patient Y?”

In some implementations, query responses (e.g., a response to the example queries above or below) may be interrogated, and information relevant to a particular patient may be requested. For example, such an input can be “What are the articles in the literature that are most relevant to patient X's condition and parameters?”

The generative artificial intelligence module 170 may also process complex inputs (e.g., inputs with multiple sentences, multiple questions, Boolean operators, etc.). For example, an initial input may be “Which healthcare members are most similar to applicant A? Show me only members from Detroit, Michigan.” Follow-up questions may include “Which plans were they recommended?” and/or “What was the range of prices associated with their plans?”

Other example inputs that may be processed/answered using generative artificial intelligence may include some or all of the following:
What is my hospital's care performance compared to other hospitals in my region?
What is the size of the current waiting list?
How many urgent patients are on the waiting list?
What is the average length of stay in medicine/orthopedics/surgery at my hospital?
What is the did not attend rate by specialty?
How many procedures are being completed each day?
How many outpatients are follow-ups and which specialty? How does that compare to the national position?
What percentage of patients on the waitlist have been validated in the past 3 months?
Where do we spend the most on agency staff?
What critical equipment and medications are low/out-of-stock?
Which clinical outcomes are not meeting national standards?
Are we carrying out any surgical procedures against NICE guidance?
In some embodiments, the generative artificial intelligence module 170 includes a personalized treatment engine. The personalized treatment engine can analyze patient-specific data models to generate customized treatment plans and use machine learning algorithms to refine and update treatment (and treatment recommendations). In one example, the personalized treatment engine may dynamically generate and adjust treatment plans based on generative AI insights.

The generative artificial intelligence module 170 may simulate and/or predict individual health scenarios to predict outcomes. The generative artificial intelligence module 170 can generate various virtual patient twins/avatars for testing potential treatments, predicting disease progression, and modeling preventive interventions. These simulations can be used to request confirmatory lab tests.

In some embodiments, the numerical representations (or, embeddings) described herein use healthcare-specific embedding technologies. In some embodiments, the embeddings may be statistical embeddings, although the artificial intelligence platform 110 may construct embeddings differently (e.g., embeddings derived from graph structures).

In some embodiments, the generative artificial intelligence module 170 provides cohorting features and functionality. This can go beyond k-nearest neighbors, for example, to look at clusters, which can improve the AI platform's ability to surface (e.g., determine) population level metrics to actually surface characteristics of various embeddings described herein.

In some embodiments, the generative artificial intelligence module 170 may automatically provide automated treatment decision without input from a physician or other user.

The AI platform 110 may also include a data integration module 172 of the AI platform 110. The generative artificial intelligence module 172 can function to ingest and/or integrate (e.g., in real-time) diverse health data types and records (genomic data, electronic health records, wearable device data, environmental data, lifestyle data) in a variety of formats (e.g., unstructured data, structured data). For example, the data integration module 172 may leverage the model-driven architecture described herein. The data integration module 172 may ensure and/or facilitate data consistency, privacy, and security while enabling real-time data updates and access.

In some embodiments, the data integration module 172 connects data sources so that embeddings (e.g., numerical representations) can be compared across disparate data sources, and relationships between embeddings can be used in data science operations.

The AI platform 110 may also include a monitoring interface module 174 of the AI platform 110. The monitoring interface module 174 can provide real-time and/or continuous health monitoring through wearable technology integration. The monitoring interface 174 can deliver real-time updates and adjustments to treatment plans and recommendations based on patient activity, biometric data, environmental changes, and the like.

The AI platform 110 may also include an error handling module 176. The error handling module 176 may function to detect and/or correct errors in recommendations (e.g., treatment recommendations) generated by the AI platform 110 prior to the recommendation being finalized and/or presented. For example, error handling module 176 can include safety violations (e.g., drug interactions). The error handling module 176 may also ensure safety conditions are satisfied. The error handling module 176 can also ensure (e.g., define and/or enforce rules) that safety conditions (e.g., allergies and other critical medical flags that must have priority over weights) are not violated. For example, even if the AI platform 110 generated a treatment recommendation that included a particular drug that a particular patient was allergic to, the error handling module 176 can intercept that recommendation based on the patient's allergy, and/or trigger an updated recommendation with the allergy as context for subsequent recommendations.

The error handling module 176 may also define and/or enforce various weight rules for different weights (e.g., model weights, data record weights, article weights) that are used to make recommendations, determine similarity values, and/or perform other functionality described herein. In some implementations, the error handling module 176 may define a priority hierarchy of the different data (e.g., health data records, electronic medical records, research and/or literature articles, journals, wearable device data, artificial intelligence insights, recommendations, and/or other data records) and ensure that the various models and functions adhere to that priority hierarchy and/or weight rules. For example, the priority may be based on the age of the data (e.g., more recent data may have a higher priority than older data). Data may also be flagged such that it will not be used at all. For example, articles that have been identified (e.g., by the medical community) as including flaws or errors (e.g., this may be inferred if an article was pulled from publication and/or if a threshold number of other articles refute the substance of that article) may be flagged and either have a lower priority and/or a NULL priority indicating that it should not be used at all.

The AI platform 110 may also include an access control module 178. The access control module 178 may enforce restrictions imposed by administrative and/or security policies, profile rights, organizational controls, etc. Access controls can be implemented at various stages of the generative artificial intelligence process to prevent particular types of information (e.g., restricted information) from being ingested by models, prompts, results, etc. For example, restricted information may be permitted to be ingested by a model (e.g., large language model, multimodal model) but the results can be omitted, suppressed, masked, or abstracted in compliance with the access control policies. The access control module 178 can use role-based access controls that can prevent users from receiving answers from the generative artificial intelligence module 170, and/or other aspects of the AI platform 110, that include information that is not commensurate with their enterprise permissions.

The access control module 178 may also define and/or enforce data retention rules. More specifically, the access control module 178 may retain and/or purge data (e.g., health data records, electronic medical records, research and/or literature articles, journals, wearable device data, artificial intelligence insights, recommendations, and/or other data records) at particular times, periods, and/or intervals. The access control module 178 may define and/or enforce different data retention rules based on the type of data (e.g., health data records, electronic medical records, research and/or literature articles, journals, wearable device data, artificial intelligence insights, recommendations), enterprise rules, and/or applicable laws (e.g., federal laws, state laws, international laws). For example, one type of data (e.g., health data records) may be retained indefinitely or for a fixed period of time (e.g., 10 years), while other types of data (e.g., journals) may be retained for a different period of time (e.g., 20 years). This can allow, for example, the models to use current data as opposed to stale data (e.g., journals over 20 years) to provide improved accuracy. This may also ensure that the systems and models are in compliance with applicable laws (e.g., privacy laws).

In a specific implementation, the access control module 178 can enforce and/or define retention rules for medical recommendations (e.g., treatment recommendations). This can increase system accuracy and efficiency (e.g., for more accurate auditing or law, policy, and/or liability considerations). In other words, the access control module 178 may enable creation of a “paper trail” so that medical recommendations can be audited, etc.

The AI platform 110 may also include an explainability module 180. The explainability module 180 may function to provide traceability and/or explainability of recommendations and/or other outputs generated by the AI platform 110. For example, the explainability module 180 can indicate portions of data records used to generate recommendations and their respective data sources. The explainability module 180 can also function to corroborate model outputs (e.g., large language model outputs). For example, the explainability module 180 can provide source citations automatically and/or on-demand to corroborate or validate model outputs. The explainability module 180 may also determine a compatibility of the different data sources that were used to generate an output. For example, the explainability module 180 may identify data records that contradict each other (e.g., one of the data records indicates that John Doe is a patient Acme hospital, and another data record indicates that John Doe is a patient at a different hospital) and provide a notification that the output was generated based on contradictory or conflicting information. In some implementations, the explainability module 180 may provide source citations with recommendations. For example, the explainability module 180 may provide source citations inline with the relevant portions (e.g., sentences) of a recommendation.

The AI platform 110 may also include a model management module 182. The model management module 182 may function to capture feedback regarding model performance (e.g., response time), model accuracy, system utilization (e.g., model processing system utilization, model processing unit utilization), and other attributes. For example, the model management module 182 can track user interactions within systems, capturing explicit feedback (e.g., through a training user interface), implicit feedback, and the like. The feedback can be used to refine models and recommendations. In some implementations, physicians may be able to provide input (e.g., feedback) on models, systems, and/or recommendation accuracy, and the model management module 182 can incorporate that feedback to retrain models, refine models, replace models, update datasets (e.g., medical datasets), and/or the like. For example, a physician may notice that a treatment recommendation is flawed, and then interrogate that recommendation to determine the sources used to generate the recommendation, and then flag one or more sources (e.g., an outdated medical article) and/or provide more relevant articles to include when generating the recommendation.

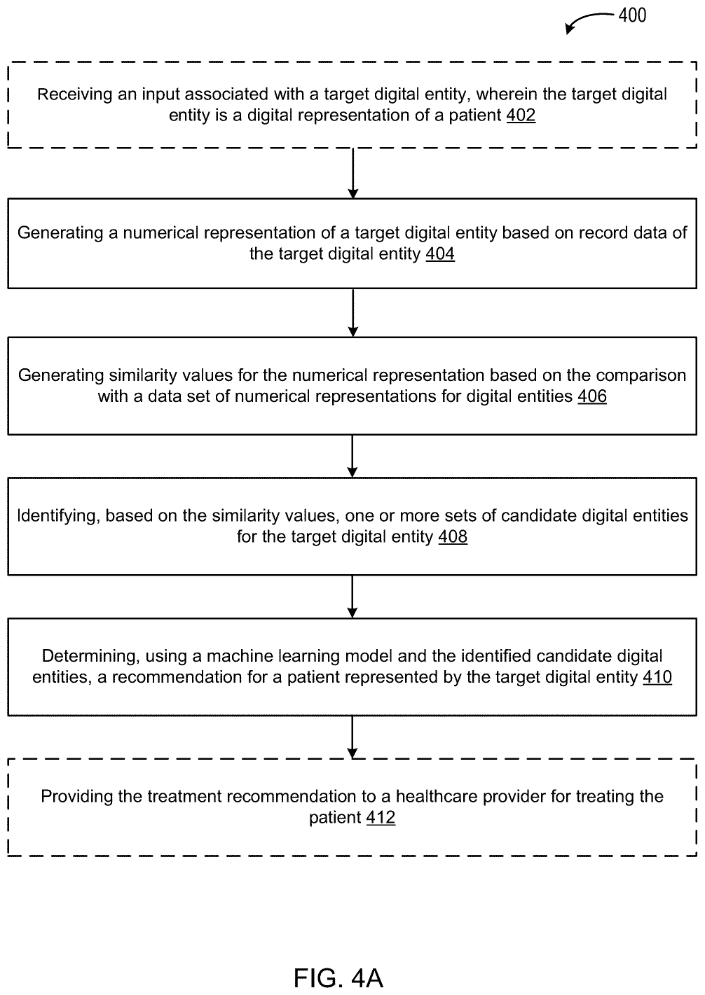

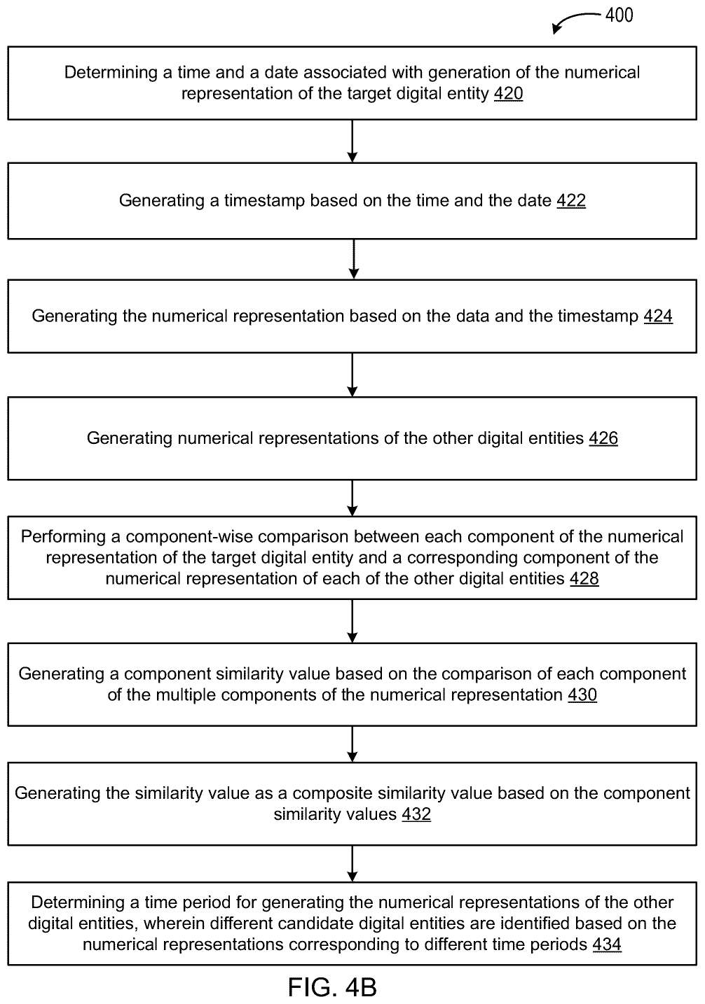

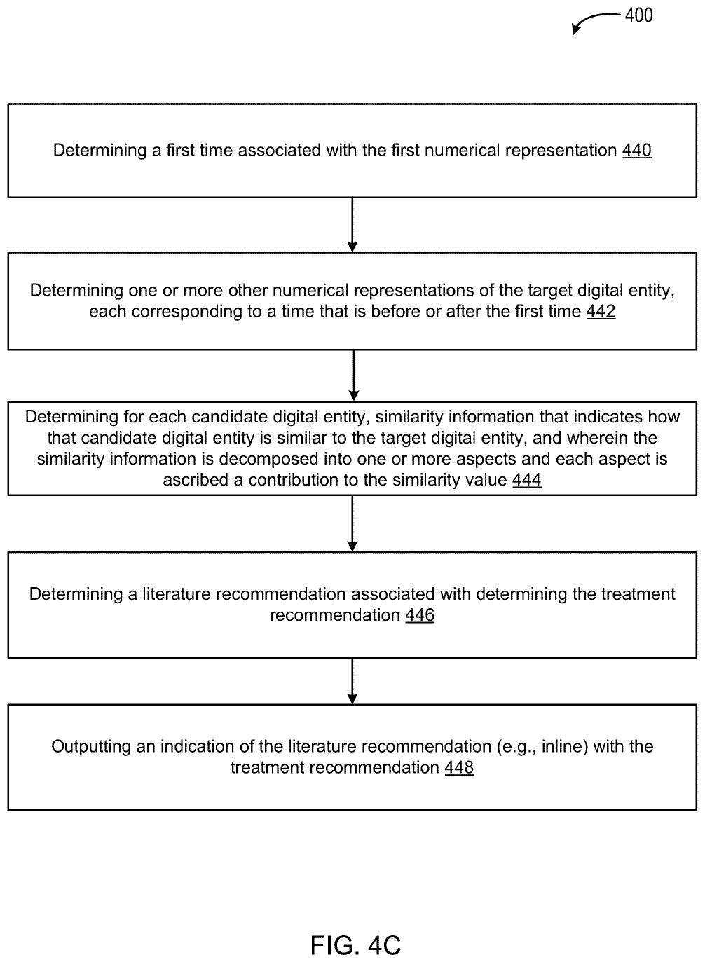

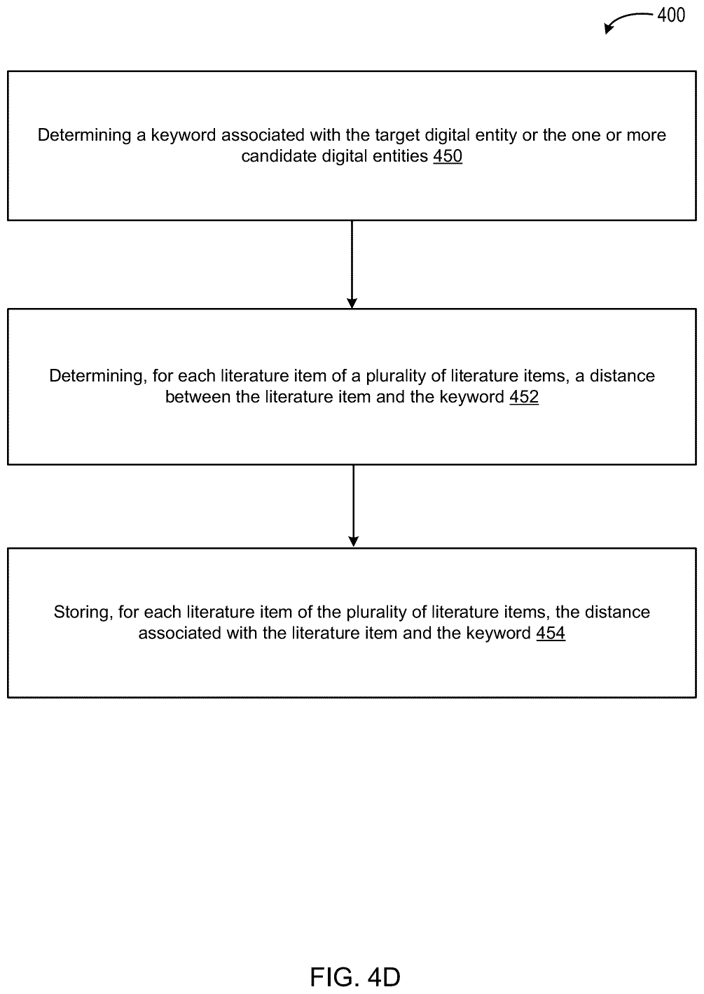

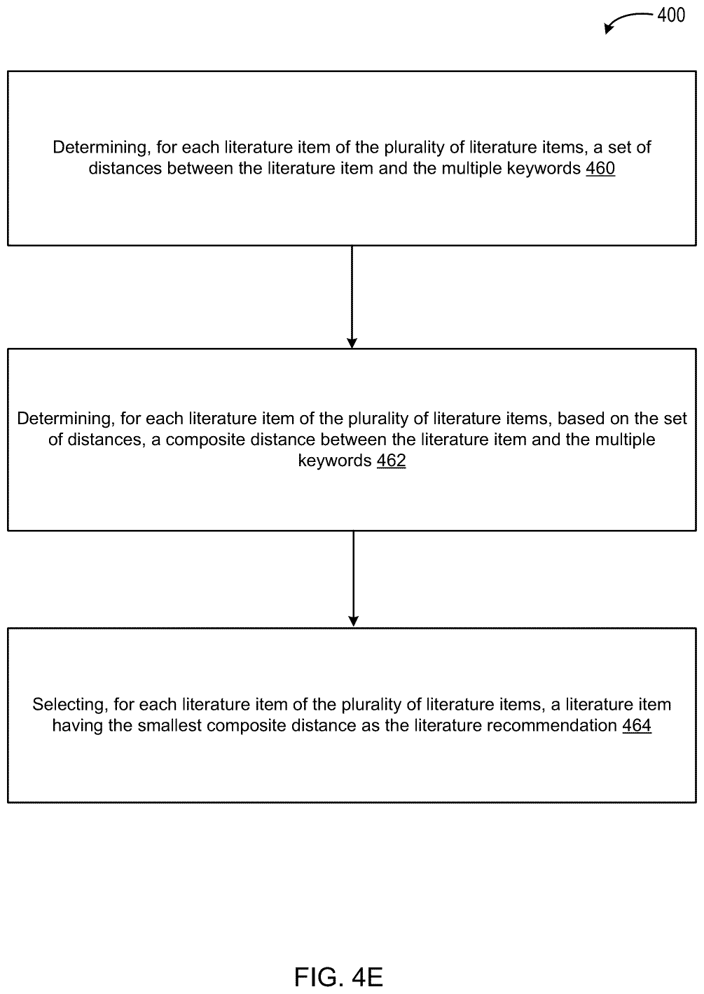

FIG. 4A depicts a flow diagram 400 of an example of a method for determining a treatment recommendation of a target digital entity using machine learning in accordance with aspects of the present disclosure. In step 402, a computing system (e.g., AI platform 110) receives an input associated with a target digital entity, wherein the target digital entity is a digital representation of a patient. The input may be a natural language query obtained from the healthcare provider treating the patient. The other digital entities may represent other patients (or other persons having medical conditions), and at least a portion of the other patients are not treated by the healthcare provider. The candidate digital entities may be identified from the other digital entities that represent the other patients. Each of the similarity values may represent a correspondence between the numerical representations of the target digital entity and the other digital entities. The machine learning model may include a multimodal model, omni-modal model, and/or a large language model (LLM), etc., and the treatment recommendation may be a natural language response to the natural language query generated by the large language model.

In some embodiments, the other digital entities may include other data that includes data categories. The data categories may include age, weight, vitals, lifestyle, prescriptions, disease status, survival status, tumor grade, tumor volume, tumor location, genetic information, single-nucleotide variant (SNV), copy number variation (CNV), treatments, chemotherapy medicine, chemotherapy dose, surgery details, surgery extent of resection, grade, diagnosis, radiology dose, concurrent radiology and chemotherapy, histopathological marker, electronic health record (EHR) information, medical record information, physician notes, lab results, test results, immunizations, medical images, environmental data, lifestyle data (or, lifestyle factors), and/or reports.

The data of the target digital entity may include electronic health records and any of proteome profiling, transcriptome profiling, methylome profiling, copy number variations, simple nucleotide variations, clinical notes, histopathology information, and radiology information. The numerical representation for the target digital entity and each of the other digital entities may include a vector having multiple components. Each component may correspond to one or more of the data categories. The at least one component of the multiple components may include multiple sub-components, and each sub-component may correspond to a different data category of the data categories or the same data category of the data categories.

In some embodiments, the data of the target digital entity includes multiple data categories, and the numerical representation of the target digital entity and/or each of the other digital entities is generated from vectors of numbers. Each vector may correspond to one or more of the categories of data. The computing system may generate the vector(s) associated with each category. More specifically, the computing system may identify data included in that category and provide that data as an input to another machine learning model and/or the same machine learning model to generate the vector encoding (or, simply, vector). Generating the vector may include normalizing the vector, and the numerical representation of the target digital entity may be based on the normalized vector. The machine learning model and/or other machine learning model may generate, for each of the other digital entities, a numerical representation of data associated with a category, and a metric is computed based on the numerical representations to capture a similarity or other relationship between one or more pairs of digital entities of other digital entities. The computing system may further process the data of the target digital entity to remove personally identifiable information associated with the target digital entity.

In step 404, the computing system generates a numerical representation of a target digital entity based on record data of the target digital entity. In step 406, the computing system generates similarity values for the numerical representation based on the comparison with a data set of numerical representations for digital entities. In step 408, the computing system identifies, based on the similarity values, one or more sets of candidate digital entities for the target digital entity. In step 410, the computing system determines, using a machine learning model (e.g., an omni-modal model) and the identified candidate digital entities, a recommendation for a patient represented by the target digital entity. In step 412, the computing system provides the treatment recommendation to a healthcare provider for treating the patient.

In some embodiments, the other digital entities may include other data that includes data categories. The data categories may include age, weight, vitals, lifestyle, prescriptions, disease status, survival status, tumor grade, tumor volume, tumor location, genetic information, single-nucleotide variant (SNV), copy number variation (CNV), treatments, chemotherapy medicine, chemotherapy dose, surgery details, surgery extent of resection, grade, diagnosis, radiology dose, concurrent radiology and chemotherapy, histopathological marker, electronic health record (EHR) information, medical record information, physician notes, lab results, test results, immunizations, medical images, environmental data, lifestyle data (or, lifestyle factors), and/or reports.

The data of the target digital entity may include electronic health records and any of proteome profiling, transcriptome profiling, methylome profiling, copy number variations, simple nucleotide variations, clinical notes, histopathology information, and radiology information. The numerical representation for the target digital entity and each of the other digital entities may include a vector having multiple components. Each component may correspond to one or more of the data categories. The at least one component of the multiple components may include multiple sub-components, and each sub-component may correspond to a different data category of the data categories or the same data category of the data categories.

In some embodiments, the data of the target digital entity includes multiple data categories, and the numerical representation of the target digital entity and/or each of the other digital entities is generated from vectors of numbers. Each vector may correspond to one or more of the categories of data. The computing system may generate the vector(s) associated with each category. More specifically, the computing system may identify data included in that category and provide that data as an input to another machine learning model and/or the same machine learning model to generate the vector encoding (or, simply, vector). Generating the vector may include normalizing the vector, and the numerical representation of the target digital entity may be based on the normalized vector. The machine learning model and/or other machine learning model may generate, for each of the other digital entities, a numerical representation of data associated with a category. The computing system may compute a metric based on the numerical representations to capture a similarity or other relationship between one or more pairs of digital entities of other digital entities. The computing system may further process the data of the target digital entity and/or other digital entities to remove personally identifiable information associated with the target digital entity and/or other digital entities.

FIG. 4B depicts a flow diagram 400 of an example of a method for generating numerical representations in accordance with aspects of the present disclosure. In step 420, the computing system determines a time and a date associated with generation of the numerical representation of the target digital entity. In step 422, the computing system generates a timestamp based on the time and the date. In step 424, the computing system generates the numerical representation based on the data and the timestamp.

In step 426, the computing system generates numerical representations of the other digital entities. The numerical representations of the other digital entities may include a first numerical representation of a particular digital entity of the other digital entities and a second numerical representation of the particular digital entity of the other digital entities. The first numerical representation may be associated with a first time and the second numerical representation may be associated with a second time that is different from the first time. The first time may correspond to a first state of a medical condition of the target digital entity and the second time may correspond to a second state of the medical condition of the target digital entity

In step 428, the computing system performs, for each component of the multiple components, a component-wise comparison between each component of the numerical representation of the target digital entity and a corresponding component of the numerical representation of each of the other digital entities.

In step 430, the computing system generates a component similarity value based on the comparison of each component of the multiple components of the numerical representation. In step 432, the computing system generates the similarity value as a composite similarity value based on the component similarity values. In step 434, the computing system determines a time period for generating the numerical representations of the other digital entities, wherein different candidate digital entities are identified based on the numerical representations corresponding to different time periods.

In some embodiments, each component is associated with a weight value of a set of weight values, and, for each of the other digital entities, generating the composite similarity value may include (e.g., for each component) multiplying the component similarity value associated with the component and the weight value associated with the component, and/or generating the composite similarity value based on a weighted sum or average of the component similarity values. The weight value may be explicitly provided by an end user, and the weight value may be inferred based on end-user preferences.

FIG. 4C depicts a flow diagram 400 of an example of a method for determining literature recommendations associated with determining the treatment recommendation in accordance with aspects of the present disclosure. In step 440, the computing system determines a first time associated with the first numerical representation. In step 442, the computing system determines one or more other numerical representations of the target digital entity, each corresponding to a time that is before or after the first time. In step 444, the computing system determines for each candidate digital entity, similarity information that indicates how that candidate digital entity is similar to the target digital entity, and wherein the similarity information is decomposed into one or more aspects and each aspect is ascribed a contribution to the similarity value. In step 446, the computing system determines a literature recommendation associated with determining the treatment recommendation, the determination of the literature recommendation being based on any of the numerical representation of the target digital entity and the numerical representations of the other candidate digital entities. In step 448, the computing system outputs an indication of the literature recommendation (e.g., inline) with the treatment recommendation.

FIG. 4D depicts a flow diagram 400 of an example of a method for determining literature recommendations in accordance with aspects of the present disclosure. In step 450, the computing system determines a keyword associated with the target digital entity or the one or more candidate digital entities. In step 452, the computing system determines, for each literature item of a literature items, a distance between the literature item and the keyword. In step 454, the computing system stores, for each literature item of the literature items, the distance associated with the literature item and the keyword.

FIG. 4E depicts a flow diagram 400 of an example of a method for determining literature recommendations in accordance with aspects of the present disclosure. In step 460, the computing system determines, for each literature item of the plurality of literature items, a set of distances between the literature item and the multiple keywords. In step 462, the computing system determines, for each literature item of the plurality of literature items, based on the set of distances, a composite distance between the literature item and the multiple keywords. In step 464, the computing system selects, for each literature item of the plurality of literature items, a literature item having the smallest composite distance as the literature recommendation. In some embodiments, some or all of the steps 460-464 may be performed for multiple keywords associated with the target digital entity or the one or more candidate digital entities.

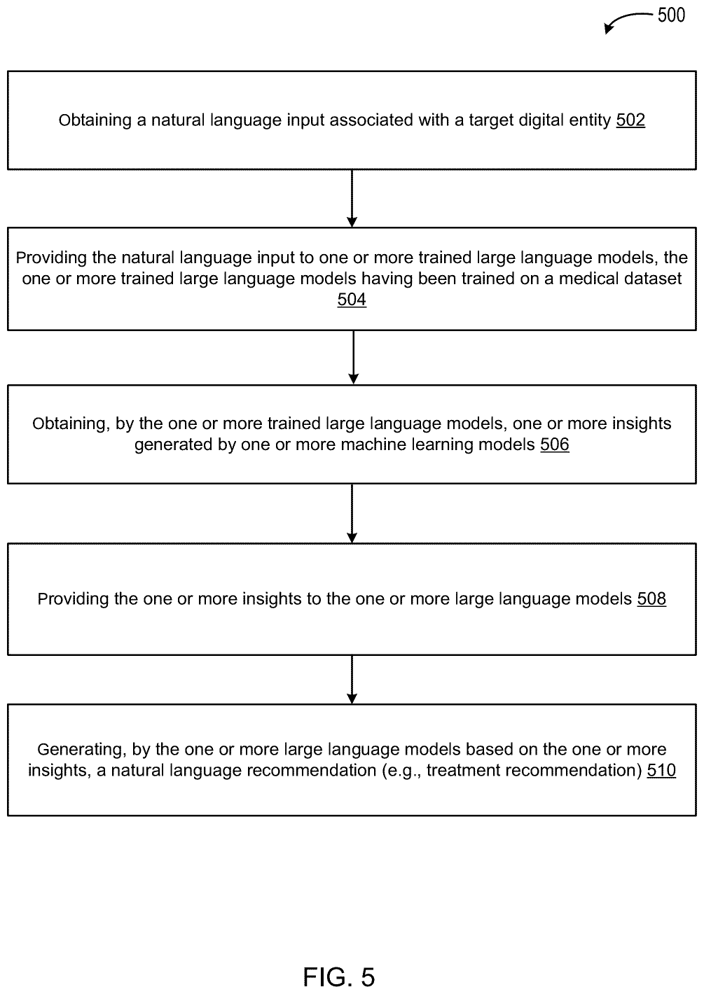

FIG. 5 depicts a flow diagram 500 of an example of a method for generating natural language recommendations using machine learning in accordance with aspects of the present disclosure. In step 502, a computing system (e.g., AI platform 110) obtains a natural language input associated with a target digital entity. The input may be a query from a healthcare provider, administrator, or insurance agent.

In step 504, the computing system provides the natural language input to one or more trained large language models, the one or more trained large language models having been trained on a medical dataset. The medical dataset may include one or more data categories. The data categories may include age, weight, vitals, lifestyle, prescriptions, disease status, survival status, tumor grade, tumor volume, tumor location, genetic information, single-nucleotide variant (SNV), copy number variation (CNV), treatments, chemotherapy medicine, chemotherapy dose, surgery details, surgery extent of resection, grade, diagnosis, radiology dose, concurrent radiology and chemotherapy, histopathological marker, electronic health record (EHR) information, medical record information, physician notes, lab results, test results, immunizations, medical images, environmental data, lifestyle data (or, lifestyle factors), and/or reports. The computing system may further train the one or more trained large language models on a dataset comprising numerical representations of a plurality of different digital entities.

In step 506, the computing system obtains, by the one or more trained large language models, one or more insights generated by one or more machine learning models. In step 508, the computing system provides the one or more insights to the one or more large language models. The input may include a query from a healthcare provider, administrator, or insurance agent. The one or more insights may have been previously generated and/or triggered in real-time based on the natural language input (e.g., query). In step 510, the computing system generates, by the one or more large language models based on the one or more insights, a natural language recommendation.

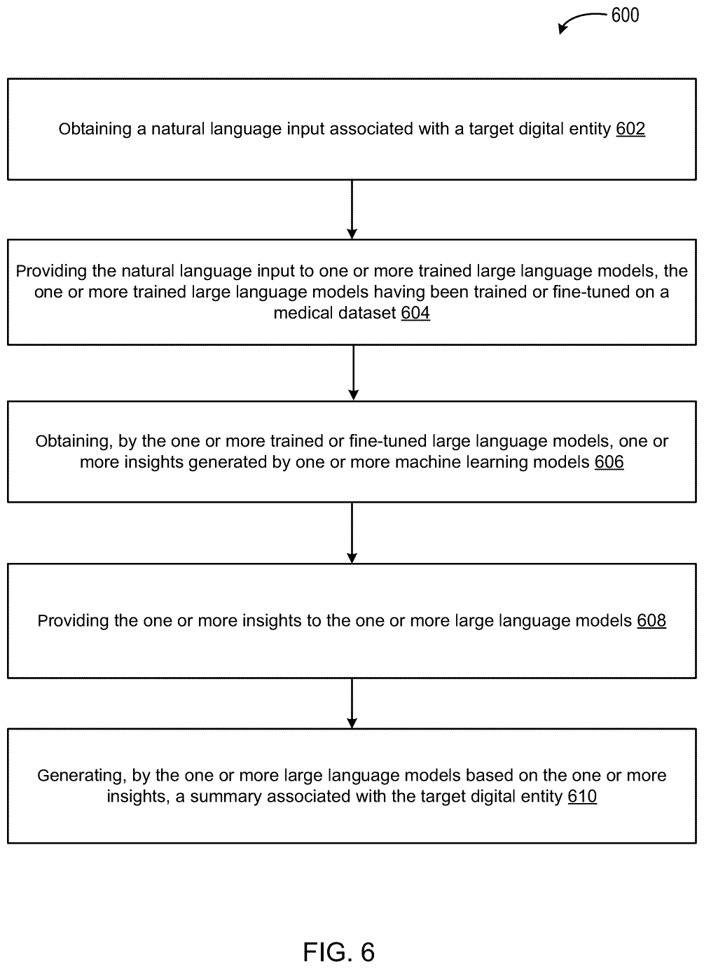

FIG. 6 depicts a flow diagram 600 of an example of a method for generating a summary of machine learning insights generated by machine learning models in accordance with aspects of the present disclosure. In step 602, a computing system (e.g., AI platform 110) obtains a natural language input associated with a target digital entity. The natural language input may be received through graphical user interface (GUI). In step 604, the computing system provides the natural language input to one or more trained large language models, the one or more trained large language models having been trained or fine-tuned on a medical dataset. In step 606, the computing system obtains, by the one or more trained or fine-tuned large language models, one or more insights generated by one or more machine learning models. In step 608, the computing system provides the one or more insights to the one or more large language models. In step 610, the computing system generates, by the one or more large language models based on the one or more insights, a summary associated with the target digital entity. The summary may include a feature breakdown of the one or more insights used to generate summary. The summary may include links to one or more literature documents associated with a medical condition of the target digital entity. In one example, at least one of literature documents was used by the one or more machine learning application to generate the one or more insights.

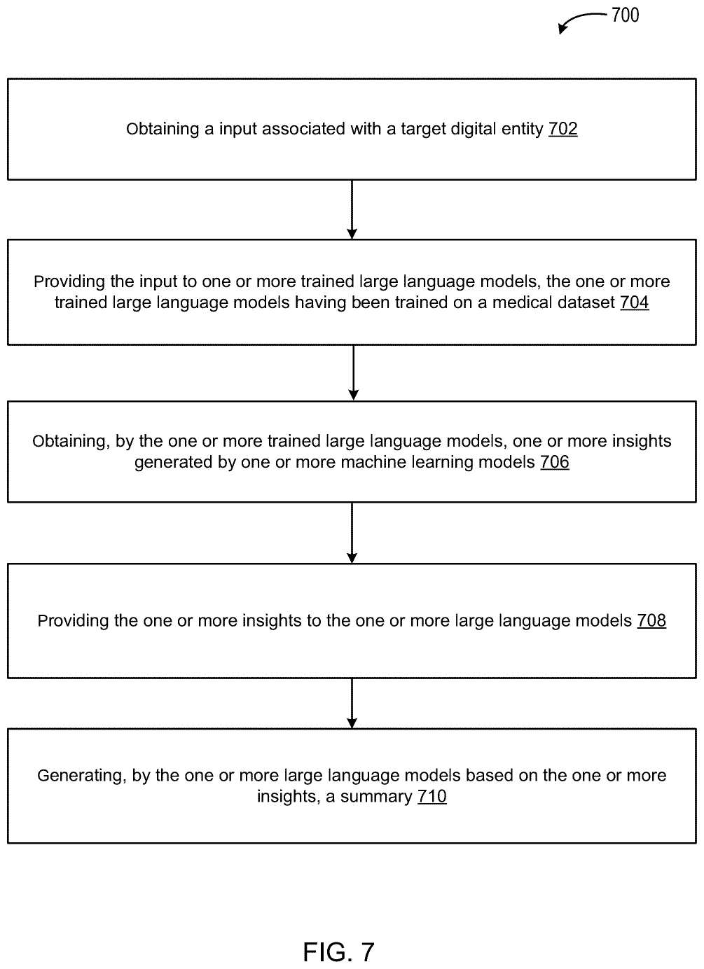

FIG. 7 depicts a flow diagram 700 of an example of a method for generating a natural language recommendation based on machine learning insights in accordance with aspects of the present disclosure. In step 702, a computing system (e.g., AI platform 110) obtains an input associated with a target digital entity. The input may be a query from a healthcare provider, administrator, or insurance agent. In step 704, the computing system provides the input to one or more trained large language models, the one or more trained large language models having been trained on a medical dataset. The medical dataset may include one or more data categories. The data categories may include age, weight, vitals, lifestyle, prescriptions, disease status, survival status, tumor grade, tumor volume, tumor location, genetic information, single-nucleotide variant (SNV), copy number variation (CNV), treatments, chemotherapy medicine, chemotherapy dose, surgery details, surgery extent of resection, grade, diagnosis, radiology dose, concurrent radiology and chemotherapy, histopathological marker, electronic health record (EHR) information, medical record information, physician notes, lab results, test results, immunizations, medical images, environmental data, lifestyle data (or, lifestyle factors), and/or reports. The computing system may further train the one or more trained large language models on a dataset comprising numerical representations of a plurality of different digital entities.

In step 706, the computing system obtains, by the one or more trained large language models, one or more insights generated by one or more machine learning models. In step 708, the computing system provides the one or more insights to the one or more large language models. The one or more insights may have been previously generated and/or triggered in real-time based on the input (e.g., query). In step 710, the computing system generates, by the one or more large language models based on the one or more insights, a summary. In some embodiments, the computing system may further train any of the one or more trained large language models and fine-tuned large language models on a dataset comprising numerical representations of a plurality of different digital entities.

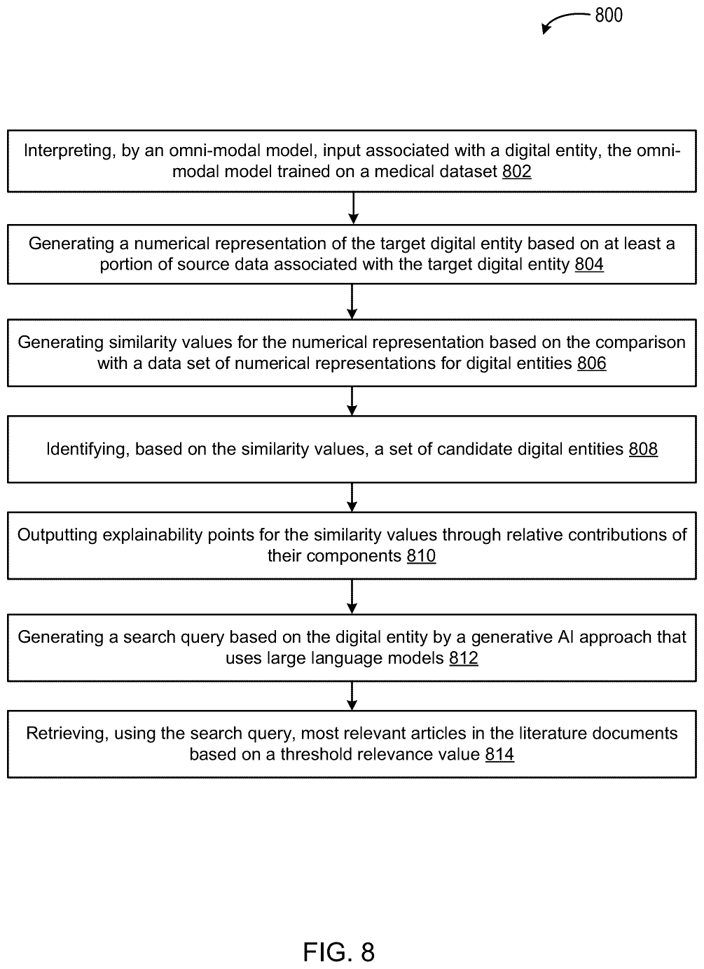

FIG. 8 depicts a flow diagram 800 of an example of a method for generating and explaining similarity values associated with a digital entity and candidate digital entities in accordance with aspects of the present disclosure. In step 802, a computing system (e.g., AI platform 110) interprets, by an omni-modal model, input associated with a digital entity. In step 804, the computing system generates a numerical representation of the target digital entity based on at least a portion of source data associated with the target digital entity. In step 806, the computing system generates similarity values for the numerical representation based on the comparison with a data set of numerical representations for digital entities. In step 808, the computing system identifies, based on the similarity values, a set of candidate digital entities. In step 810, the computing system outputs explainability points for the similarity values through relative contributions of their components. Explainability points provide end-user trust, model auditability, and responsible AI implementation. Explainability points can be output for understanding model decisions, characterizing model accuracy and biases, as well as expected impact and potential biases.

In step 812, the computing system generates a search query based on the digital entity by a generative AI approach that uses large language models. In step 814, the computing system retrieves, using the search query, most relevant articles in the literature documents based on a threshold relevance value. In some embodiments, some or all of the steps 804-814 may be performed in response to interpreting the input.

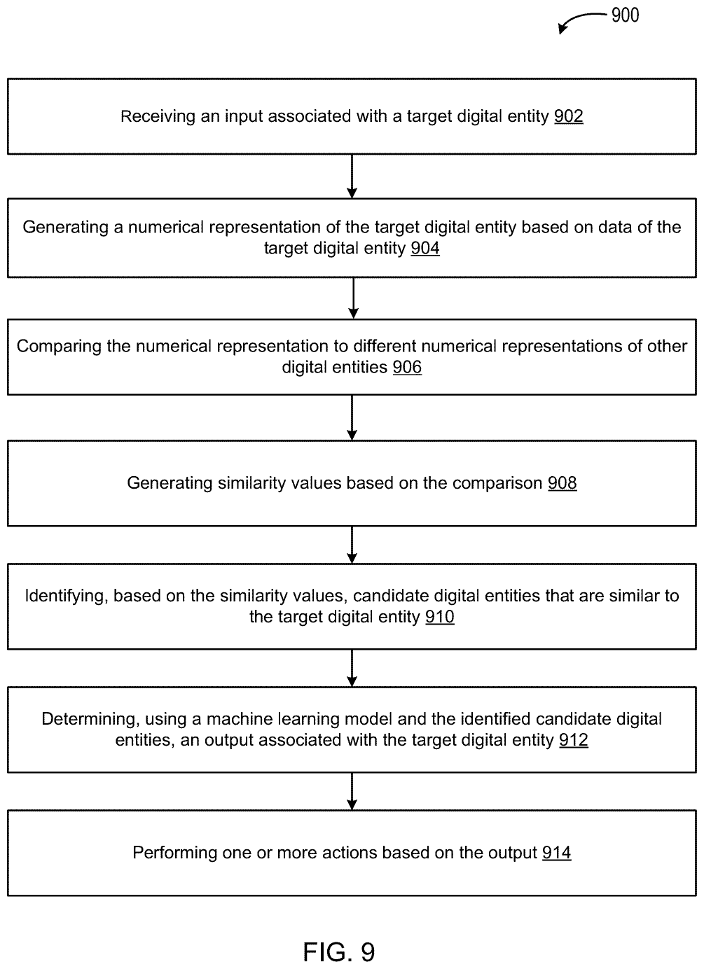

FIG. 9 depicts a flow diagram 900 of an example of a method for identifying candidate digital entities that are similar to a target digital entity and determining an output based on the candidate digital entities using machine learning in accordance with aspects of the present disclosure. In step 902, a computing system (e.g., AI platform 110) receives an input associated with a target digital entity. In step 904, the computing system generates a numerical representation of the target digital entity based on data of the target digital entity. In step 906, the computing system compares the numerical representation to different numerical representations of other digital entities. In step 908, the computing system generates similarity values based on the comparison. In step 910, the computing system identifies, based on the similarity values, candidate digital entities that are similar to the target digital entity. In step 912, the computing system determines, using a machine learning model and the identified candidate digital entities, an output associated with the target digital entity. In step 914, the computing system performs one or more actions based on the output.

It is noted that the components, functional blocks, and modules described herein with respect to FIGS. 1-9 may include or be executed by processors, electronics devices, hardware devices, electronics components, logical circuits, memories, software codes, firmware codes, among other examples, or any combination thereof. In addition, features discussed herein may be implemented via specialized processor circuitry, via executable instructions, or combinations thereof.

In the context of the present disclosure a model-driven architecture is a term for a software design approach that provides models as a set of guidelines for structuring specifications. An example model-driven architecture may include a type system that may be used as a domain-specific language (DSL) within a platform used to access data, interact with data, and/or perform processing or analytics based on one or more type or function definitions within the type system. By using an abstraction layer provided by a type system, the complexity of a logistics optimization application problem can be reduced by orders of magnitude, such as to the order of a few thousand types, for any given logistics optimization application that a programmer manipulates using JAVASCRIPT or another language to achieve a desired result. Thus, all of the complexity of the underlying foundation (with an order of M×S×T×A×U using structured programming paradigms) is abstracted and simplified for the programmer. Here, M represents the number of process modules (APACHE Open Source modules are examples of process modules), S represents the number of disparate enterprise and extraprise data sources, T represents the number of unique sensored devices, A represents the number of programmatic APIs, and U represents the number of user presentations or interfaces. Example technologies that can be included in one or more embodiments may include nearly-free and unlimited compute capacity and storage in scale-out cloud environments, such as AMAZON Web Services (AWS); big data and real-time streaming; smart connected devices; mobile computing; and data science including big-data analytics and machine learning to process the volume, velocity, and variety of big-data streams.

In some embodiments, the data integration and machine learning-based data modules may function to obtain electronic data from a variety of disparate data sources over a communication network. The disparate data sources may include internal data sources and external data sources. The internal data sources may be internal relative to the data integration and machine learning-based modules discussed herein. The external data sources may be data sources that are external relative to the data integration and machine learning-based modules and/or an entity associated with the data integration and machine learning-based modules.

The external data sources may represent a multitude of possible sources of data relevant to the functionality disclosed herein. The external data may include, for example, external document repositories or access systems, API connectors, point of sale (POS) systems, databases, external model pipelines, distributor systems, Information Resources, Inc, (IRI) systems, Internet systems, reference intelligence systems, and/or the like. In various embodiments, functionality of the external data source systems may be performed by one or more servers (e.g., a cloud-based server) and/or other computing devices.

The systems, methods, engines, datastores, and/or databases described herein may be at least partially processor-implemented, with a particular processor or processors being an example of hardware. For example, at least some of the operations of a method may be performed by one or more processors or processor-implemented engines. Moreover, the one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., an Application Program Interface (API)).

The performance of certain of the operations may be distributed among the processors, not only residing within a single machine, but deployed across a number of machines. In some example embodiments, the processors or processor-implemented engines may be located in a single geographic location (e.g., within a home environment, an office environment, or a server farm). In other example embodiments, the processors or processor-implemented engines may be distributed across a number of geographic locations.

The type system of the model-driven architecture may include types as data objects and at least one of: associated methods, associated logic, and associated machine learning models. One or more of the data objects may be associated with at least one of: one or more customers, one or more companies, one or more accounts, one or more products, one or more employees, one or more suppliers, one or more opportunities, one or more contracts, one or more locations, and one or more digital portals. Type definitions may include properties or characteristics of an implemented software construct.

Employing the type system of the model-driven architecture may include performing data modeling to translate raw source data formats into target types. Sources of data may be associated with at least one of: electronic health records, physicians, patients, encounters, health care plans, member details, claim history, accounts, products, employees, suppliers, opportunities, contracts, locations, digital portals, geolocation manufacturers, supervisory control and data acquisition (SCADA) information, open manufacturing system (OMS) information, inventories, supply chains, bills of materials, transportation services, maintenance logs, and service logs, and/or other sources of data described herein. Among other things, the type system can be employed to perform data modeling in order to translate raw source data formats into target types. Sources of data for which data modeling and translation can be performed may include accounts, products, employees, suppliers, opportunities, contracts, locations, digital portals, geolocation manufacturers, SCADA information, OMS information, inventories, supply chains, bills of materials, transportation services, maintenance logs, or service logs.

The model-driven architecture enables capabilities and applications including precise predictive analytics, massively parallel computing at the edge of a network, and fully-connected sensor networks at the core of a business value chain. The model-driven architecture can serve as the nerve center that connects and enables collaboration among previously-separate business functions, including product development, marketing, sales, service support, manufacturing, inventory, finance, shipping, order management, human capital management, etc. Some embodiments may include a product cloud that includes software running on a hosted elastic cloud technology infrastructure that stores or processes product data, customer data, enterprise data, and Internet data. The product cloud may provide one or more of: a platform for building and processing software applications; massive data storage capacity; a data abstraction layer that implements a type system; a rules engine and analytics platform; a machine learning engine; smart product applications; and social human-computer interaction models. One or more of the layers or services may depend on the data abstraction layer for accessing stored or managed data, communicating data between layers or applications, or otherwise storing, accessing, or communicating data.

The model-driven architecture may operate as a comprehensive design, development, provisioning, and operating platform for industrial-scale applications in various industries, such as energy industries, health or healthcare, wearable technology industries, sales and advertising industries, transportation industries, communication industries, scientific and geological study industries, military and defense industries, financial services industries, healthcare industries, manufacturing industries, retail, government organizations, and/or the like. The system may enable integration and processing of large and highly dynamic data sets from enormous networks and large-scale information systems.

An integration component, data services component, and modular services component may store, transform, communicate, and process data based on the type system. In some embodiments, the data sources and/or the applications may also operate based on the type system. In an example embodiment, the applications may be configured to operate or interface with the components based on the type system. For example, the applications may include business logic written in code and/or accessing types defined by a type system to leverage services provided by the system.

In some embodiments, the model-driven architecture uses a type system that provides type-relational mapping based on a plurality of defined types. For example, the type system may define types for use in the applications, such as a type for a customer, organization, device, or the like. During development of an application, an application developer may write code that accesses the type system to read or write data to the system, perform processing or business logic using defined functions, or otherwise access data or functions within defined types. In some embodiments, the model-driven architecture enforces validation of data or type structure using annotations/keywords. The types in the type system may include defined view configuration types used for rendering type data on a screen in a graphical, text, or other format. In some embodiments, a server, such as a server that implements at least a portion of the system, may implement mapping between data stored in one or more databases and a type in the type system, such as data that corresponds to a specific customer type or other type.

One example of a type system is given by way of the following non-limiting example, which may be used in various embodiments and in combination with any other teachings of this disclosure. In some embodiments, the fundamental concept in the type system is a “type,” which is similar to a “class” in object-oriented programming languages. At least one difference between “class” in some languages and “type” in some embodiments of the type system disclosed here is that the type system is not tied to any particular programming language. As discussed here, at least some embodiments disclosed here include a model-driven architecture, where types are the models. Not only are types interfaces across different underlying technologies, but they are also interfaces across different programming languages. In fact, the type system can be considered self-describing, so below is presented an overview of the types that may define the type system itself.

A type is the definition of a potentially-complex object that the system understands. Types may be the primary interface for all platform services and the primary way that application logic is organized. Some types are defined by and built into the platform itself. These types provide a uniform model across a variety of underlying technologies. Platform types also provide convenient functionality and build up higher-level services on top of low-level technologies. Other types are defined by the developers using the platform. Once installed in the environment, they can be used in the same ways as the platform types. There is no sharp distinction between types provided by the platform and types developed using the platform.

The logistics optimization application can be used with various enterprise functions, such as messaging, reporting, alerting, etc., processes to update systems based on triggers, detecting anomalies, real-time data streams, etc. In example enterprise environments, the logistics optimization application can control or instruct manufacturing or resource planning systems.

In an example implementation when volatility is predicted or detected in supply chains networks, simulations using hyperparameters and uncertainty distributions can be employed to automatically realize instructions or commands that can re-strategize to better leverage manufacturing, such as minimizing the amount of inventory held in a supply chain.

An example model-driven architecture for integrating, processing, and abstracting data related to an enterprise logistics optimization application development platform can include tools for machine learning, application development and deployment, data visualization, automated control and instruction, other tools (such as an integration component, a data services component, a modular services component, and an application that may be located on or behind an application layer), etc.

Those of skill will further appreciate that the various illustrative logical blocks, modules, circuits, and algorithm steps described in connection with the disclosure herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure. Skilled artisans will also readily recognize that the order or combination of components, methods, or interactions that are described herein are merely examples and that the components, methods, or interactions of the various aspects of the present disclosure may be combined or performed in ways other than those illustrated and described herein.

The various illustrative logics, logical blocks, modules, circuits, and algorithm processes described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and processes described above. Whether such functionality is implemented in hardware or software depends upon the particular application and design constraints imposed on the overall system.

The hardware and data processing apparatus used to implement the various illustrative logics, logical blocks, modules, and circuits described in connection with the aspects disclosed herein may be implemented or performed with a general purpose single- or multi-chip processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, or any conventional processor, controller, microcontroller, or state machine. In some implementations, a processor may also be implemented as a combination of computing devices, such as a combination of a DSP and a microprocessor, a plurality of microprocessors, one or more microprocessors in conjunction with a DSP core, or any other such configuration. In some implementations, particular processes and methods may be performed by circuitry that is specific to a given function.

In one or more aspects, the functions described may be implemented in hardware, digital electronic circuitry, computer software, firmware, including the structures disclosed in this specification and their structural equivalents thereof, or any combination thereof. Implementations of the subject matter described in this specification also may be implemented as one or more computer programs, that is one or more modules of computer program instructions, encoded on a computer storage media for execution by, or to control the operation of, data processing apparatus.

If implemented in software, the functions may be stored on or transmitted over as one or more instructions or code on a computer-readable medium. The processes of a method or algorithm disclosed herein may be implemented in a processor-executable software module which may reside on a computer-readable medium. Computer-readable media includes both computer storage media and communication media including any medium that may be enabled to transfer a computer program from one place to another. A storage media may be any available media that may be accessed by a computer. By way of example, and not limitation, such computer-readable media can include random-access memory (RAM), read-only memory (ROM), electrically erasable programmable read-only memory (EEPROM), CD-ROM or other optical disk storage, magnetic disk storage or other magnetic storage devices, or any other medium that may be used to store desired program code in the form of instructions or data structures and that may be accessed by a computer. Also, any connection may be properly termed a computer-readable medium. Disk and disc, as used herein, includes compact disc (CD), laser disc, optical disc, digital versatile disc (DVD), floppy disk, hard disk, solid state disk, and Blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and instructions on a machine readable medium and computer-readable medium, which may be incorporated into a computer program product.

Various modifications to the implementations described in this disclosure may be readily apparent to those skilled in the art. Accordingly, it is to be understood that the principles and concepts defined herein may be applied via other implementations and such other implementations should be deemed to fall within the scope of this disclosure. Thus, the claims are not intended to be limited to the implementations shown herein, but are to be accorded the widest scope consistent with this disclosure, as well as the novel and inventive principles and features disclosed herein.

Additionally, a person having ordinary skill in the art will readily appreciate, the terms “upper” and “lower” are sometimes used for ease of describing the figures, and indicate relative positions corresponding to the orientation of the figure on a properly oriented page, and may not reflect the proper orientation of any device as implemented.

Certain features that are described in this specification in the context of separate implementations also may be implemented in combination in a single implementation. Conversely, various features that are described in the context of a single implementation also may be implemented in multiple implementations separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. Further, the drawings may schematically depict one more example processes in the form of a flow diagram. However, other operations that are not depicted may be incorporated in the example processes that are schematically illustrated. For example, one or more additional operations may be performed before, after, simultaneously, or between any of the illustrated operations. In certain circumstances, multitasking and parallel processing may be advantageous. Moreover, the separation of various system components in the implementations described above should not be understood as requiring such separation in all implementations, and it should be understood that the described program components and systems may generally be integrated together in a single software product or packaged into multiple software products. Additionally, some other implementations are within the scope of the following claims. In some cases, the actions recited in the claims may be performed in a different order and still achieve desirable results.

As used herein, including in the claims, various terminology is for the purpose of describing particular implementations only and is not intended to be limiting of implementations. For example, as used herein, an ordinal term (e.g., “first,” “second,” “third,” etc.) used to modify an element, such as a structure, a component, an operation, etc., does not by itself indicate any priority or order of the element with respect to another element, but rather merely distinguishes the element from another element having a same name (but for use of the ordinal term). The term “coupled” is defined as connected, although not necessarily directly, and not necessarily mechanically; two items that are “coupled” may be unitary with each other. the term “or,” when used in a list of two or more items, means that any one of the listed items may be employed by itself, or any combination of two or more of the listed items may be employed. For example, if a composition is described as containing components A, B, or C, the composition may contain A alone; B alone; C alone; A and B in combination; A and C in combination; B and C in combination; or A, B, and C in combination. Also, as used herein, including in the claims, “or” as used in a list of items prefaced by “at least one of” indicates a disjunctive list such that, for example, a list of “at least one of A, B, or C” means A or B or C or AB or AC or BC or ABC (that is A and B and C) or any of these in any combination thereof. The term “substantially” is defined as largely but not necessarily wholly what is specified—and includes what is specified; e.g., substantially 90 degrees includes 90 degrees and substantially parallel includes parallel—as understood by a person of ordinary skill in the art. In any disclosed aspect, the term “substantially” may be substituted with “within [a percentage] of” what is specified, where the percentage includes 0.1, 1, 5, and 10 percent; and the term “approximately” may be substituted with “within 10 percent of” what is specified. The phrase “and/or” means and or.

Various embodiments of the present disclosure include systems (e.g., having one or more processors, and memory storing instructions that, when executed by the one or more processors, cause the system to perform functionality described herein), methods, and non-transitory computer-readable medium (or media) configured to perform generating a numerical representation of a target digital entity based on record data of the target digital entity; generating similarity values for the numerical representation based on a comparison with a data set of numerical representations for digital entities; identifying, based on the similarity values, one or more sets of candidate digital entities for the target digital entity; determining, using a machine learning model and the identified candidate digital entities, a recommendation for a patient represented by the target digital entity.

In some embodiments, an input associated with the target digital entity is received, and the input is a natural language query obtained from the healthcare provider treating the patient. In some embodiments, the other digital entities represent other patients, wherein at least a portion of the other patients are not treated by the healthcare provider. In some embodiments, the candidate digital entities are identified from the other digital entities that represent the other patients. In some embodiments, each of the similarity values represent a correspondence between the numerical representations of the target digital entity and the other digital entities. In some embodiments, the machine learning model includes an omni-modal model, and the treatment recommendation comprises a natural language response to the natural language query generated by the large language model.

In some embodiments, the other digital entities includes other data that include data categories, the data categories including any of age, weight, vitals, lifestyle, prescriptions, disease status, survival status, tumor grade, tumor volume, tumor location, genetic information, single-nucleotide variant, copy number variation, treatments, chemotherapy medicine, chemotherapy dose, surgery details, surgery extent of resection, grade, diagnosis, radiology dose, concurrent radiology and chemotherapy, histopathological marker, electronic health record (EHR) information, medical record information, physician notes, lab results, test results, immunizations, medical images and reports. In some embodiments, the data of the target digital entity includes electronic health records and any of proteome profiling, transcriptome profiling, methylome profiling, copy number variations, simple nucleotide variations, clinical notes, histopathology information, and radiology information.

In some embodiments, the numerical representation for the target digital entity and each of the other digital entities include a vector having multiple components, and each component may correspond to one or more of the data categories. At least one component of the multiple components may include multiple sub-components, and each sub-component of the multiple sub-components corresponds to a different data category of the data categories or the same data category of the data categories. In some embodiments, the data of the target digital entity includes multiple data categories, and wherein the numerical representation of the target digital entity and each of the other digital entities is generated from vectors of numbers, wherein each vector corresponds to one or more of the categories of data. In some embodiments, generating the vector associated with each category includes identifying data included in that category, and providing that data as an input to another machine learning model to generate the vector encoding.

The systems, methods, and non-transitory computer-readable medium (or media) may be further configured to perform processing the data of the target digital entity to remove personally identifiable information associated with the target digital entity. In some embodiments, generating the vector includes normalizing the vector, and wherein the numerical representation of the target digital entity is based on the normalized vector.

In some embodiments, the other machine learning model generates, for each of the other digital entities, a numerical representation of data associated with a category, and a metric is computed based on the numerical representations to capture a similarity or other relationship between one or more pairs of digital entities of other digital entities. In some embodiments, generating the numerical representation of the target digital entity includes determining a time and a date associated with generation of the numerical representation of the target digital entity; generating a timestamp based on the time and the date; and generating the numerical representation based on the data and the timestamp.

The systems, methods, and non-transitory computer-readable medium (or media) may be further configured to perform generating numerical representations of the other digital entities, wherein the representations of the other digital entities include a first numerical representation of a particular digital entity of the other digital entities and a second numerical representation of the particular digital entity of the other digital entities, and wherein the first numerical representation is associated with a first time and the second numerical representation is associated with a second time that is different from the first time. In some embodiments, the first time corresponds to a first state of a medical condition of the target digital entity and the second time corresponds to a second state of the medical condition of the target digital entity.

In some embodiments, comparing the numerical representation of the target digital entity to the numerical representations of the other digital entities includes performing, for each component of the multiple components, a component-wise comparison between each component of the numerical representation of the target digital entity and a corresponding component of the numerical representation of each of the other digital entities; generating a component similarity value based on the comparison of each component of the multiple components of the numerical representation; and generating the similarity value as a composite similarity value based on the component similarity values.

In some embodiments, each component is associated with a weight value of a set of weight values, and wherein, for each of the other digital entities, generating the composite similarity value includes, for each component, multiplying the component similarity value associated with the component and the weight value associated with the component; and generating the composite similarity value based on a weighted sum or average of the component similarity values.

The systems, methods, and non-transitory computer-readable medium (or media) may be further configured to perform determining a time period for generating the numerical representations of the other digital entities, wherein different candidate digital entities are identified based on the numerical representations corresponding to different time periods.

The systems, methods, and non-transitory computer-readable medium (or media) may be further configured to perform determining a first time associated with the first numerical representation; and determining one or more other numerical representations of the target digital entity, each corresponding to a time that is before or after the first time.

The systems, methods, and non-transitory computer-readable medium (or media) may be further configured to perform determining for each candidate digital entity, similarity information that indicates how that candidate digital entity is similar to the target digital entity, and wherein the similarity information is decomposed into one or more aspects and each aspect is ascribed a contribution to the similarity value.

The systems, methods, and non-transitory computer-readable medium (or media) may be further configured to perform determining a literature recommendation associated with determining the treatment recommendation, the determination of the literature recommendation being based on any of the numerical representation of the target digital entity and the numerical representations of the other candidate digital entities; and outputting an indication of the literature recommendation inline with the treatment recommendation. In some embodiments, determining the literature recommendation includes determining a keyword associated with the target digital entity or the one or more candidate digital entities; for each literature item of a plurality of literature items: determining a distance between the literature item and the keyword; and storing the distance associated with the literature item and the keyword. In some embodiments, determining the literature recommendation includes for multiple keywords associated with the target digital entity or the one or more candidate digital entities: for each literature item of the plurality of literature items: determining a set of distances between the literature item and the multiple keywords; and determining, based on the set of distances, a composite distance between the literature item and the multiple keywords; and selecting a literature item having the smallest composite distance as the literature recommendation.

Various embodiments of the present disclosure include systems (e.g., having one or more processors, and memory storing instructions that, when executed by the one or more processors, cause the system to perform functionality described herein), methods, and non-transitory computer-readable medium (or media) configured to perform obtaining a natural language input associated with a target digital entity; providing the natural language input to one or more trained large language models, the one or more trained large language models having been trained on a medical dataset; obtaining, by the one or more trained large language models, one or more insights generated by one or more machine learning models; providing the one or more insights to the one or more large language models; and generating, by the one or more large language models based on the one or more insights, a natural language recommendation.

In some embodiments, the medical dataset includes one or more data categories, the one or more data categories including any of age, weight, vitals, lifestyle, prescriptions, disease status, survival status, tumor grade, tumor volume, tumor location, genetic information, single-nucleotide variant, copy number variation, treatments, chemotherapy medicine, chemotherapy dose, surgery details, surgery extent of resection, grade, diagnosis, radiology dose, concurrent radiology and chemotherapy, histopathological marker, electronic health record information, medical record information, physician notes, lab results, test results, immunizations, medical images and reports.

In some embodiments, the input comprises a query from a healthcare provider, administrator, or insurance agent. In some embodiments, the one or more insights have been previously generated. In some embodiments, the natural language input triggered generation of the one or more insights. The systems, methods, and non-transitory computer-readable medium (or media) may be further configured to perform training the one or more trained large language models on a dataset comprising numerical representations of a plurality of different digital entities.

Various embodiments of the present disclosure include systems (e.g., having one or more processors, and memory storing instructions that, when executed by the one or more processors, cause the system to perform functionality described herein), methods, and non-transitory computer-readable medium (or media) configured to perform obtaining a natural language input associated with a target digital entity; providing the natural language input to one or more trained large language models, the one or more trained large language models having been trained or fine-tuned on a medical dataset; obtaining, by the one or more trained or fine-tuned large language models, one or more insights generated by one or more machine learning models; providing the one or more insights to the one or more large language models; and generating, by the one or more large language models based on the one or more insights, a summary associated with the target digital entity.

In some embodiments, the summary includes a feature breakdown of the one or more insights used to generate summary. In some embodiments, the summary includes links to one or more literature documents associated with a medical condition of the target digital entity, wherein at least one of the one or more literature documents was used by the one or more machine learning application to generate the one or more insights. In some embodiments, the natural language input comprises a natural language input.

Various embodiments of the present disclosure include systems (e.g., having one or more processors, and memory storing instructions that, when executed by the one or more processors, cause the system to perform functionality described herein), methods, and non-transitory computer-readable medium (or media) configured to perform obtaining an input associated with a target digital entity; providing the input to one or more trained large language models, the one or more trained large language models having been trained on a medical dataset; obtaining, by the one or more trained large language models, one or more insights generated by one or more machine learning models; providing the one or more insights to the one or more large language models; and generating, by the one or more large language models based on the one or more insights, a summary.

In some embodiments, the medical dataset includes one or more data categories, the one or more data categories including any of age, weight, vitals, lifestyle, prescriptions, disease status, survival status, tumor grade, tumor volume, tumor location, genetic information, single-nucleotide variant, copy number variation, treatments, chemotherapy medicine, chemotherapy dose, surgery details, surgery extent of resection, grade, diagnosis, radiology dose, concurrent radiology and chemotherapy, histopathological marker, electronic health record information, medical record information, physician notes, lab results, test results, immunizations, medical images and reports. In some embodiments, input comprises a query from a healthcare provider, administrator, or insurance agent. In some embodiments, the one or more insights have been previously generated. In some embodiments, the input triggered generation of the one or more insights.

The systems, methods, and non-transitory computer-readable medium (or media) may be further configured to perform training any of the one or more trained large language models and fine-tuned large language models on a dataset comprising numerical representations of a plurality of different digital entities.

Various embodiments of the present disclosure include systems (e.g., having one or more processors, and memory storing instructions that, when executed by the one or more processors, cause the system to perform functionality described herein), methods, and non-transitory computer-readable medium (or media) configured to perform obtaining a natural language input associated with a target digital entity; providing the natural language input to one or more trained large language models, the one or more trained large language models having been any of trained and fine-tuned on a medical dataset; obtaining, by the one or more trained large language models, one or more insights generated by one or more machine learning models; providing the one or more insights to the one or more large language models; and generating, by the one or more large language models based on the one or more insights, a natural language summary associated with the target digital entity.

In some embodiments, the summary includes a feature breakdown of the one or more insights used to generate the natural language summary. In some embodiments, the summary includes links to one or more literature documents associated with a medical condition of the target digital entity, wherein at least one of the one or more literature documents was used by the one or more machine learning application to generate the one or more insights. In some embodiments, the natural language input comprises a natural language input.

Various embodiments of the present disclosure include systems (e.g., having one or more processors, and memory storing instructions that, when executed by the one or more processors, cause the system to perform functionality described herein), methods, and non-transitory computer-readable medium (or media) configured to perform interpreting, by an omni-modal model, input associated with a digital entity, the omni-modal model trained on a medical dataset; in response to the interpreting: generating a numerical representation of the target digital entity based on at least a portion of source data associated with the target digital entity; generating similarity values for the numerical representation based on the comparison with a data set of numerical representations for digital entities; identifying, based on the similarity values, a set of candidate digital entities; and outputting explainability points for the similarity values through relative contributions of their components.

In some embodiments, a weight value is explicitly provided by an end user, and the weight value can be inferred based on end-user preferences. In some embodiments, a search query is generated based on the target digital entity by a generative AI approach that uses omni-modal models, and wherein the search query is used to retrieve most relevant articles in literature documents based on a threshold relevance value.

Various embodiments of the present disclosure include systems (e.g., having one or more processors, and memory storing instructions that, when executed by the one or more processors, cause the system to perform functionality described herein), methods, and non-transitory computer-readable medium (or media) configured to perform receiving an input associated with a target digital entity; generating a numerical representation of the target digital entity based on data of the target digital entity; comparing the numerical representation to different numerical representations of other digital entities; generating similarity values based on the comparison; identifying, based on the similarity values, candidate digital entities that are similar to the target digital entity; and determining, using a machine learning model and the identified candidate digital entities, an output associated with the target digital entity; and performing one or more actions based on the output.

Although the aspects of the present disclosure and their advantages have been described in detail, it should be understood that various changes, substitutions and alterations can be made herein without departing from the spirit of the disclosure as defined by the appended claims. Moreover, the scope of the present application is not intended to be limited to the particular implementations of the process, machine, manufacture, composition of matter, means, methods and processes described in the specification. As one of ordinary skill in the art will readily appreciate from the present disclosure, processes, machines, manufacture, compositions of matter, means, methods, or operations, presently existing or later to be developed that perform substantially the same function or achieve substantially the same result as the corresponding aspects described herein may be utilized according to the present disclosure. Accordingly, the appended claims are intended to include within their scope such processes, machines, manufacture, compositions of matter, means, methods, or operations.

1. A method comprising:
generating a numerical representation of a target digital entity based on record data of the target digital entity;
generating similarity values for the numerical representation based on a comparison with a data set of numerical representations for digital entities;
identifying, based on the similarity values, one or more sets of candidate digital entities for the target digital entity;
determining, using a machine learning model and the identified candidate digital entities, a recommendation for a patient represented by the target digital entity.

2. The method of claim 1, wherein an input associated with the target digital entity is received, and the input is a natural language query obtained from the healthcare provider treating the patient.

3. The method of claim 1, wherein the other digital entities represent other patients, wherein at least a portion of the other patients are not treated by the healthcare provider.

4. The method of claim 1, wherein the candidate digital entities are identified from the other digital entities that represent the other patients.

5. The method of claim 1, wherein each of the similarity values represent a correspondence between the numerical representations of the target digital entity and the other digital entities.

6. The method of claim 2, wherein the machine learning model includes an omni-modal model, and the treatment recommendation comprises a natural language response to the natural language query generated by the large language model.

7. The method of claim 1, wherein the other digital entities includes other data that include data categories, the data categories including any of age, weight, vitals, lifestyle, prescriptions, disease status, survival status, tumor grade, tumor volume, tumor location, genetic information, single-nucleotide variant (SNV), copy number variation (CNV), treatments, chemotherapy medicine, chemotherapy dose, surgery details, surgery extent of resection, grade, diagnosis, radiology dose, concurrent radiology and chemotherapy, histopathological marker, electronic health record (EHR) information, medical record information, physician notes, lab results, test results, immunizations, medical images and reports.

8. The method of claim 1, wherein the data of the target digital entity includes electronic health records and any of proteome profiling, transcriptome profiling, methylome profiling, copy number variations, simple nucleotide variations, clinical notes, histopathology information, and radiology information.

9. The method of claim 7, wherein the numerical representation for the target digital entity and each of the other digital entities include a vector having multiple components, and wherein each component corresponds to one or more of the data categories.

10. The method of claim 9, wherein at least one component of the multiple components includes multiple sub-components, and each sub-component of the multiple sub-components corresponds to a different data category of the data categories or the same data category of the data categories.

11. The method of claim 1, wherein the data of the target digital entity includes multiple data categories, and wherein the numerical representation of the target digital entity and each of the other digital entities is generated from vectors of numbers, wherein each vector corresponds to one or more of the categories of data.

12. The method of claim 11, wherein generating the vector associated with each category includes identifying data included in that category, and providing that data as an input to another machine learning model to generate the vector encoding.

13. The method of claim 12, further comprising processing the data of the target digital entity to remove personally identifiable information associated with the target digital entity.

14. The method of claim 13, wherein generating the vector includes normalizing the vector, and wherein the numerical representation of the target digital entity is based on the normalized vector.

15. The method of claim 12, wherein the other machine learning model generates, for each of the other digital entities, a numerical representation of data associated with a category, and a metric is computed based on the numerical representations to capture a similarity or other relationship between one or more pairs of digital entities of other digital entities.

16. The method of claim 1, wherein generating the numerical representation of the target digital entity includes:
determining a time and a date associated with generation of the numerical representation of the target digital entity;
generating a timestamp based on the time and the date; and
generating the numerical representation based on the data and the timestamp.

17. The method of claim 9, further comprising:
generating numerical representations of the other digital entities, wherein the representations of the other digital entities include a first numerical representation of a particular digital entity of the other digital entities and a second numerical representation of the particular digital entity of the other digital entities, and wherein the first numerical representation is associated with a first time and the second numerical representation is associated with a second time that is different from the first time.

18. The method of claim 17, wherein the first time corresponds to a first state of a medical condition of the target digital entity and the second time corresponds to a second state of the medical condition of the target digital entity.

19. A method comprising:
obtaining a natural language input associated with a target digital entity;
providing the natural language input to one or more trained large language models, the one or more trained large language models having been trained on a medical dataset;
obtaining, by the one or more trained large language models, one or more insights generated by one or more machine learning models;
providing the one or more insights to the one or more large language models; and
generating, by the one or more large language models based on the one or more insights, a natural language recommendation.

20. A method comprising:
interpreting, by an omni-modal model, input associated with a digital entity, the omni-modal model trained on a medical dataset;
in response to the interpreting:
generating a numerical representation of the target digital entity based on at least a portion of source data associated with the target digital entity;
generating similarity values for the numerical representation based on the comparison with a data set of numerical representations for digital entities;
identifying, based on the similarity values, a set of candidate digital entities; and
outputting explainability points for the similarity values through relative contributions of their components.
